(19) United States
(12) Patent Application Publication
(10) Pub. No.: US 2020/0197546 A1
(43) Pub. Date: Jun. 25, 2020

(54) USE OF ANTI-B7H3 ANTIBODIES FOR TREATING CANCER IN THE CENTRAL NERVOUS SYSTEM

(71) Applicants: MEMORIAL SLOAN-KETTERING CANCER CENTER, New York, NY (US); Y-MABS THERAPEUTICS, INC., New York, NY (US)

(72) Inventors: Kim Kramer, New York, NY (US); Cheung Nai-Kong, New York, NY (US); Ole Baadsgaard, Hellerup (DK); Claus J. Møller San-Pedro, København Ø (DK)

(73) Assignees: MEMORIAL SLOAN-KETTERING CANCER CENTER, New York, NY (US); Y-MABS THERAPEUTICS, INC., New York, NY (US)

(21) Appl. No.: 16/613,028

(22) PCT Filed: May 14, 2018
(86) PCT No.: PCT/US2018/032559

§ 371 Date: Nov. 12, 2019

Related U.S. Application Data
(60) Provisional application No. 62/505,558, filed on May 12, 2017.

(51) Int. Cl. A61K 51/10; C07K 16/28; A61P 35/00

(52) U.S. Cl. CPC A61K 51/1045; A61P 35/00; C07K 16/2827; A61K 51/1096

(57) ABSTRACT

The presently disclosed subject matter provides uses of anti-B7H3 antibodies for treating cancers in the central nervous system (CNS), including tumors metastatic to CNS, and in particular leptomeningeal carcinomatosis.

### CROSS-REFERENCE TO RELATED APPLICATION

This application claims priority to U.S. Provisional Application No. 62/505,558 filed on May 12, 2017, the contents of which are incorporated by reference in their entirety, and to which priority is claimed.

### SEQUENCE LISTING

The specification incorporates by reference the Sequence Listing submitted via EFS on May 14, 2018. Pursuant to 37 C.F.R. § 1.52(e)(5), the Sequence Listing text file, identified as 0727340700SL, is 21,716 bytes in size and was created on May 14, 2018. The Sequence Listing, electronically filed on May 14, 2018, does not extend beyond the scope of the specification and thus does not contain new matter.

### FIELD OF THE INVENTION

The presently disclosed subject matter relates to uses of anti-B7H3 antibodies for treating cancer in the central nervous system (CNS), including tumors metastatic to CNS, and in particular leptomeningeal carcinomatosis.

### BACKGROUND OF THE INVENTION

Adult CNS tumors include primary CNS tumors (formed by cancerous cells arising within the CNS) and tumors metastatic to CNS (cancer cells spread to the CNS from primary tumors originating in other organs in the body). About 20,000 new cases of primary CNS tumors are diagnosed in the U.S. each year, and an estimated 24-45% of all cancer patients in the U.S. have brain metastases. The leptomeninges (the inner two membranes enveloping the brain and spinal cord) has emerged as a sanctuary metastatic site leading to relapse. Leptomeningeal metastasis (LM; also referred to as leptomeningeal carcinomatosis) occurs when tumor cells gain access to cerebrospinal fluid pathways, travel to distant sites within the brain and spinal cord, settle, and grow. LM has been widely assumed to be invariably fatal.

Primary CNS tumors are the third most common cancer occurring among adolescents and young adults (ages 15-39) and the third most common cause of cancer death in this age group. Metastatic CNS tumors, on the other hand, are most common in adults than children. Therefore, there remains a need for innovative treatment for adult CNS tumors.

### DETAILED DESCRIPTION OF DRAWINGS

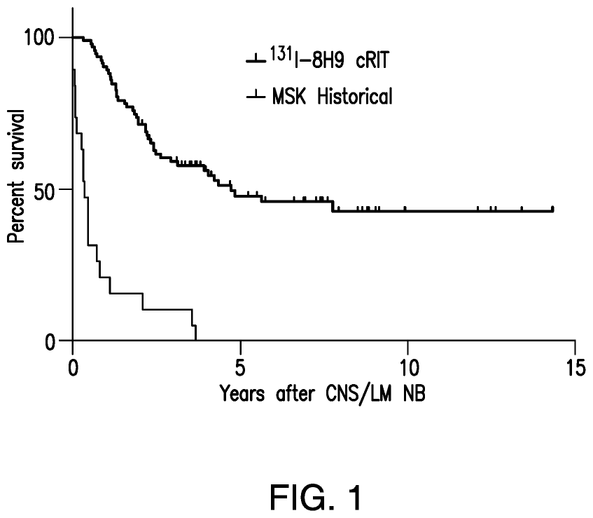

FIG. 1. Survival of pediatric neuroblastoma patients treated at MSK pre cRIT 8H9 compared with that of all ¹³¹I-8H9-treated patients.

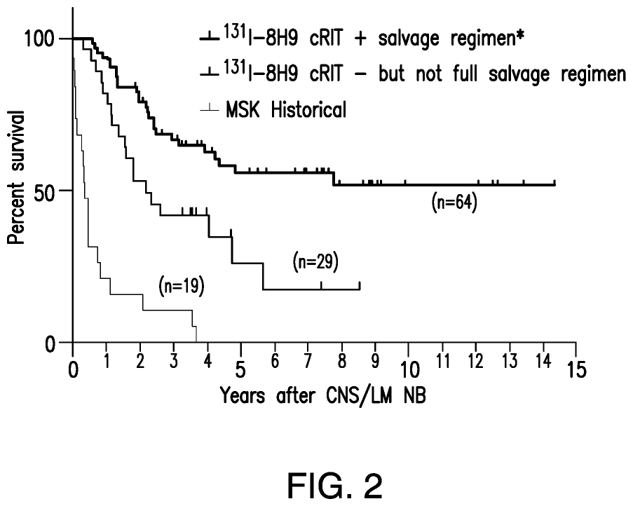

FIG. 2. Survival of pediatric neuroblastoma patients treated at MSK before 2003 compared with that of ¹³¹I-8H9-treated patients. The survival of 64 patients treated with full CNS directed therapy (blue line), 29 patients treatment with ¹³¹I-8H9 and other therapies (red line) and 19 patients treated at MSK before initiation of the protocol in 2003 (purple line) were compared with Kaplan-Meier analysis.

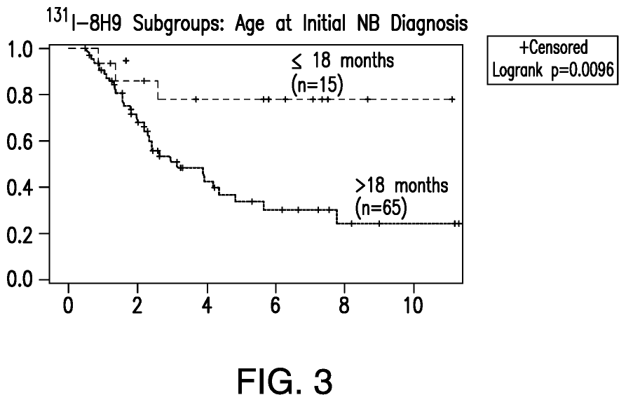

FIG. 3. Survival based on Age at Initial Neuroblastoma Diagnosis.

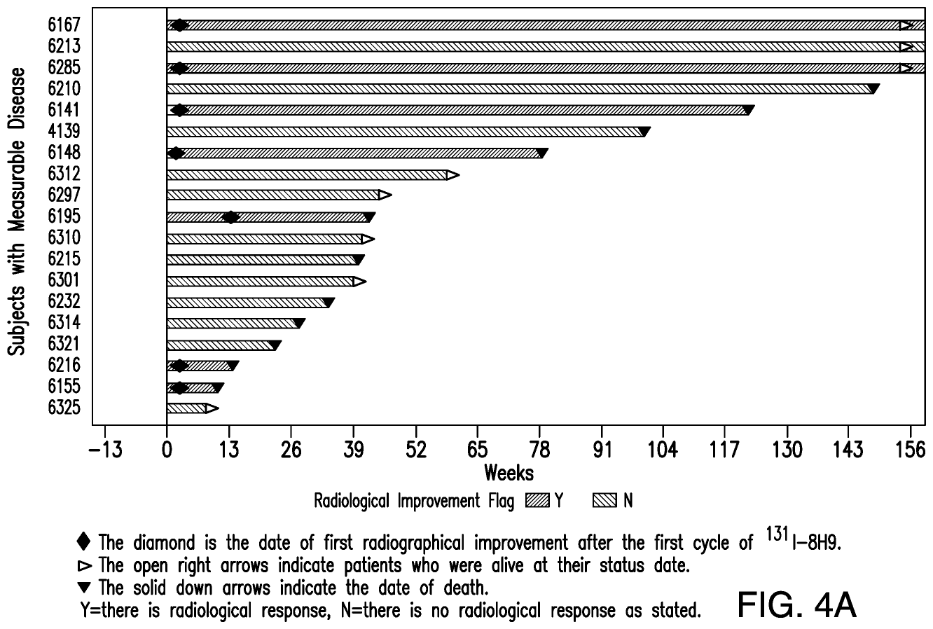

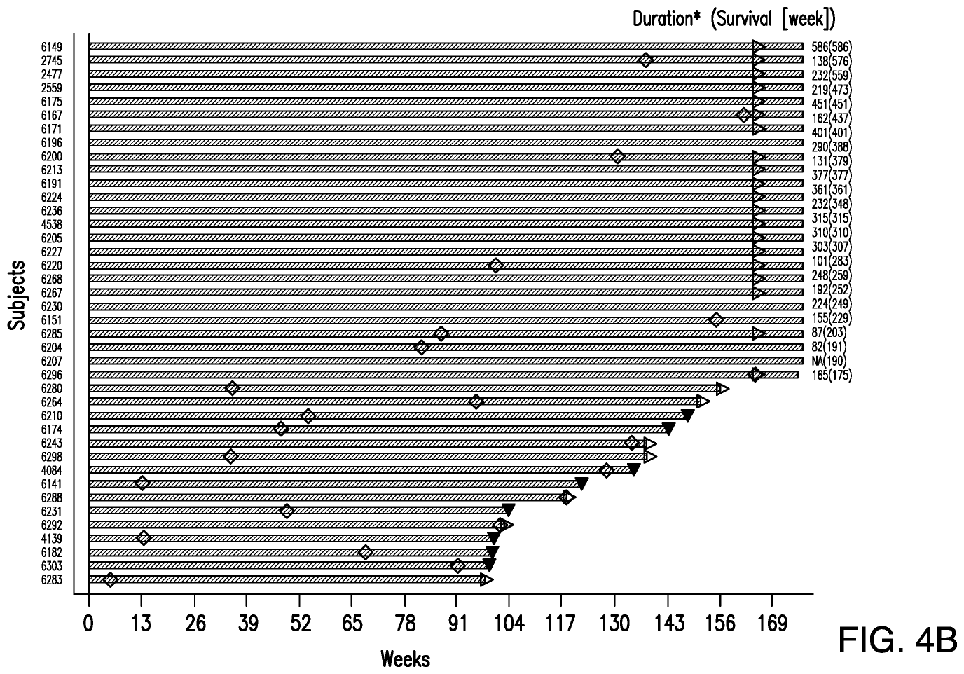

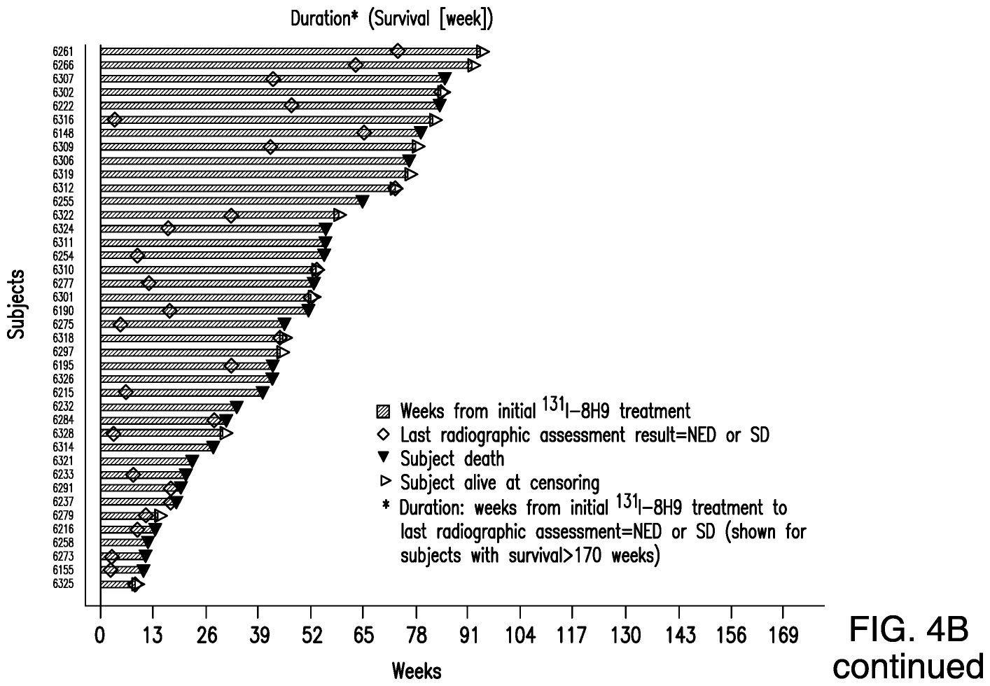

FIGS. 4A-4B. Time to first radiographical improvement in groups of patients (4A and 4B) with measurable disease at study entry. The length of the horizontal bars equates to the duration of a subject's participation in Protocol 03-133 or in post-study follow-up. Radiographical improvement was determined by comparison of pre-treatment scans with post-treatment scans. Improvement is defined as a complete response, partial response, or no evidence of disease. ♦ The diamond is the date of first radiographical improvement after the first cycle of 131I-8H9. [symbol] The open right arrows indicate patients who were alive at their status date. ▾ The solid down arrows indicate the date of death. Y axis represents individual patient, X axis represents time. Y=there is radiological response, N=there is no radiological response as stated.

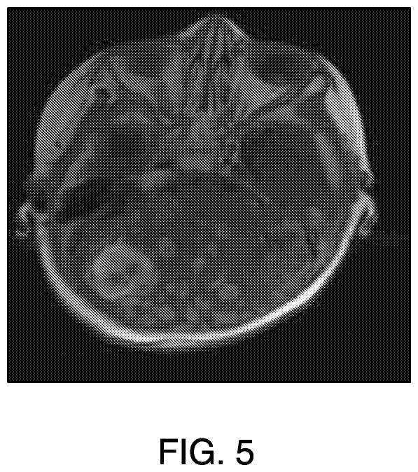

FIG. 5. Multifocal Focal CNS Neuroblastoma in remission for >7 years. CNS relapse demonstrating innumerable supratentorial, infratentorial and spinal metastases.

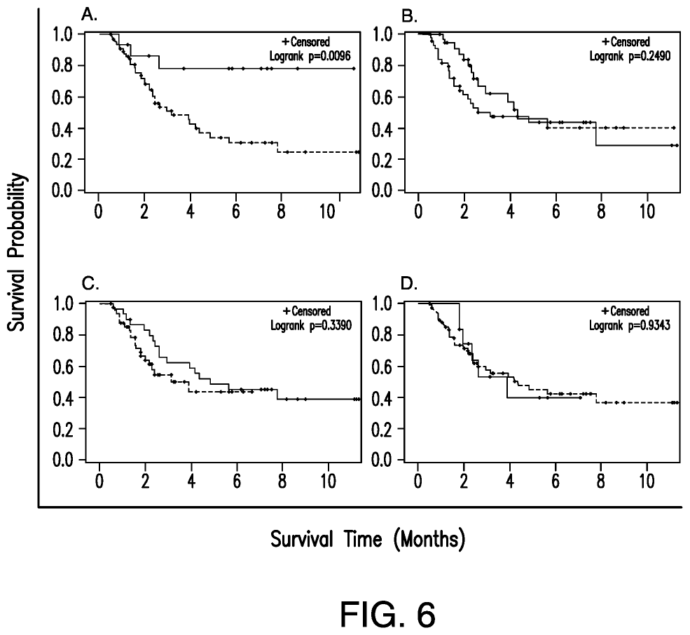

FIG. 6. Subgroup analyses and effects on overall patient survival. The effects of specific variables on overall patient survival were assessed by Kaplan-Meier analyses of subgroups of patients treated with ¹³¹I-8H9. (A) The effect of age on survival was assessed in patients ≤18 months (blue line) and in patients >18 months (red line) at initial neuroblastoma diagnosis. (B) The effect of MYCN status on survival was assessed in ¹³¹I-8H9-treated patients with amplified MYCN (blue line) and in patients with non-amplified MYCN (red line) tumors. (C) The effect of era of enrollment on the protocol on survival was assessed in patients who enrolled from 2003-2009 (blue line) and in patients who enrolled from 2010-2016 (red line) on the protocol. (D) The effect of prior CSI therapy on survival was assessed in patients who were not treated with CSI (blue line) and in patients who were treated with CSI (red line) before ¹³¹I-8H9 cRIT.

### SUMMARY OF THE INVENTION

The presently disclosed subject matter relates to uses of anti-B7H3 antibodies for treating CNS cancers, including primary CNS cancers and cancers metastatic to the CNS. In particular embodiments, anti-B7H3 antibodies are administered into the CNS to treat leptomeningeal metastasis of a cancer in an adult subject.

In certain non-limiting embodiments, the presently disclosed subject matter provides methods for treating a cancer in a human subject, comprising administering, into the CNS of the subject, a therapeutically effective amount of an antibody or an antigen-binding fragment thereof that specifically binds to B7H3. In certain embodiments, the cancer is a primary CNS cancer or a cancer metastatic to the CNS. In certain embodiments, the antibody or antigen-binding fragment thereof is conjugated to a radioactive isotope and/or atherapeutic modality (e.g., chelator compound or anticancer agent). In certain embodiments, the human subject is an adult. In certain embodiments, the cancer is metastatic to the leptomeninges. In certain embodiments, the cancer metastatic to the CNS is a solid tumor arising outside of the CNS. In certain embodiments, the solid tumor is selected from the group consisting of sarcoma, melanoma, ovarian cancer, and rhabdomyosarcoma. In certain embodiments, the solid tumor is selected from the group consisting of melanoma, ovarian cancer, and rhabdomyosarcoma. In certain embodiments, the central nerve system (CNS) cancer is selected from the group consisting of neuroblastoma and primary recurrent CNS malignancies. In certain embodiments, the cancer metastatic to the CNS is a solid tumor selected from the group consisting of breast cancer (for example, triple-negative breast cancer), and lung cancer (for example, small cell lung cancer and non-small cell lung cancer). In certain embodiments, the antibody or antigen-binding fragment is, is derived from, and/or is structurally related to, 8H9, including, but not limited to, murine, humanized, chimeric and human versions of 8H9 (see below).

In certain non-limiting embodiments, the presently disclosed subject matter provides methods for treating a cancer in a human subject, comprising administering to the subject, a therapeutically effective amount of an antibody or an antigen-binding fragment thereof, conjugated to a radioactive isotope and/or other therapeutic modality, that specifically binds to human B7H3. In certain embodiments, the cancer is any cancer that comprises B7H3 positive cancer or tumor cells. In certain embodiments, the cancer is primary to, or metastatic to, the CNS of the subject and the antibody is administered into the CNS of the subject. In certain embodiments, the subject is an adult. In certain embodiments, the subject is not an adult.

In certain non-limiting embodiments, the antibody or antigen-binding fragment thereof that specifically binds to human B7H3 is selected from the group consisting of murine antibodies or antigen-binding fragments thereof, humanized antibodies or antigen-binding fragments thereof, chimeric antibodies or antigen-binding fragments thereof, and human antibodies or antigen-binding fragments thereof. In certain embodiments, the antibody is a murine antibody or antigen-binding fragments thereof. In certain embodiments, the antibody or antigen-binding fragment thereof binds to FG-loop of B7H3. In certain embodiments, the antibody or antigen-binding fragment is, is derived from, and/or is structurally related to, 8H9, including, but not limited to, murine, humanized, chimeric and human versions of 8H9 (see below).

In certain embodiments, the antibody or antigen-binding fragment thereof comprises: (a) a heavy chain variable region CDR1 comprising the amino acid sequence set forth in SEQ ID NO: 3 (NYDIN), (b) a heavy chain variable region CDR2 comprising the amino acid sequence set forth in SEQ ID NO: 4 (WIFPGDGSTQY), (c) a heavy chain variable region CDR3 comprising the amino acid sequence set forth in SEQ ID NO: 5 (QTTATWFAY), (d) a light chain variable region CDR1 comprising the amino acid sequence set forth in SEQ ID NO: 6 (RASQSISDYLH), (e) a light chain variable region CDR2 comprising the amino acid sequence set forth in SEQ ID NO: 7 (YASQSIS), and/or (f) a light chain variable region CDR3 comprising the amino acid sequence set forth in SEQ ID NO: 8 (QNGHSFPLT). In certain embodiments, the antibody or antigen-binding fragment thereof comprises: (a) a heavy chain variable region comprising the amino acid sequence set forth in SEQ ID NO: 1, and/or (b) a light chain variable region comprising the amino acid sequence set forth in SEQ ID NO: 2.

In certain embodiments, the antibody or antigen-binding fragment thereof comprises at least: (a) a heavy chain variable region CDR comprising the amino acid sequence set forth in SEQ ID NO: 3 (NYDIN), SEQ ID NO: 4 (WIFPGDGSTQY), or SEQ ID NO: 5 (QTTATWFAY), and (b) a light chain variable region CDR comprising the amino acid sequence set forth in SEQ ID NO: 6 (RASQSISDYLH), SEQ ID NO: 7 (YASQSIS), or SEQ ID NO: 8 (QNGHSFPLT).

In certain embodiments, the antibody or antigen-binding fragment thereof is administered intrathecally to the subject. In certain embodiments, the antibody or antigen-binding fragment thereof is administered to the subject via an intraventricular device. In certain embodiments, the intraventricular device is an intraventricular catheter. In certain embodiments, the intraventricular device is an intraventricular reservoir.

In certain embodiments, the radioactive isotope that is conjugated with the antibody or fragment is ¹²⁴I, ¹³¹I, ¹⁷⁷Lu, or ^{99m}Tc

In certain embodiments, the presently disclosed methods further comprise administering to the subject one treatment cycle of the antibody or antigen-binding fragment thereof. In certain embodiments, the methods comprise administering to the subject two treatment cycles of the antibody or antigen-binding fragment thereof. In certain embodiments, one treatment cycle comprises a dosimetry dose and a treatment dose. In certain embodiments, the therapeutically effective amount is about 10 mCi to about 200 mCi. In certain embodiments, the therapeutically effective amount is about 50 mCi.

In certain embodiments, the method prolongs survival of the subject relative to a control subject or control subject population not receiving the treatment. In certain embodiments, the method prolongs remission of the cancer in the subject relative to a control subject or control subject population not receiving the treatment.

In certain embodiments, the antibody or antigen-binding fragment thereof comprises an amino acid sequence having at least about 80%, about 90%, about 95%, about 99% or about 100% homologous to the amino acid sequence set forth in SEQ ID NO: 17. In certain embodiments, the antibody or antigen-binding fragment thereof comprises the amino acid sequence set forth in SEQ ID NO: 17. In certain embodiments, the antibody or antigen-binding fragment thereof has amino acids 224-241 of SEQ ID NO: 17. In certain embodiments, the antibody or antigen-binding fragment thereof has amino acids 242-267 of SEQ ID NO: 17.

In certain embodiments, the therapeutic modality is selected from the group consisting of one or more chelator compound, one or more chemotherapeutic agent, one or more checkpoint inhibitor agent, and radiation therapy. In certain embodiments, a therapeutic modality that is not a radioactive isotope is conjugated to the antibody. In certain embodiments, the therapeutic modality is a chelator compound. In certain embodiments, a radioactive isotope is indirectly bound to the antibody or antigen-binding fragment thereof via a chelator compound, e.g. DOTA, DOTA-like compound, or DTPA. In certain embodiments, the antibody or antigen-binding fragment thereof is conjugated to a chelator compound, wherein the chelator compound is bound to a radioactive isotope. In certain embodiments, the chelator compound is DOTA or DTPA. In certain embodiments, the antibody or antigen-binding fragment thereof is conjugated to a chelator compound (e.g. DOTA, DTPA, or a related compound) and the chelator is bound, in vitro or in vivo, to radioactive isotope (e.g. ¹²⁴I, ¹³¹I, ¹⁷⁷Lu, or ^{99m}Tc).

In certain embodiments, the therapeutic modality is a monoclonal antibody 3F8 (MoAb 3F8), a granulocyte-macrophage-colony-stimulating factor (GM-CSF), or a combination thereof. In certain embodiments, the therapeutic modality is administered into the CNS of the subject and/or systemically to the subject. In certain embodiments, the therapeutic modality is administered to the subject concurrently or sequentially with the antibody or antigen-binding fragment thereof.

In another aspect, the present disclosure provides an antibody or an antigen-binding fragment thereof binding specifically to human B7H3, wherein the antibody or antigen-binding fragment thereof is conjugated to a chelator compound, wherein the chelator compound is bound to a radioactive isotope.

In certain embodiments, the antibody is selected from the group consisting of murine antibodies and antigen-binding fragments thereof, humanized antibodies and antigen-binding fragments thereof, chimeric antibodies and antigen-binding fragments thereof, and human antibodies and antigen-binding fragments thereof. In certain embodiments, the antibody or antigen-binding fragment thereof is a murine antibody or an antigen-binding fragment thereof. In certain embodiments, the antibody or antigen-binding fragment thereof binds to FG-loop of B7H3.

In certain embodiments, the antibody or antigen-binding fragment thereof comprises: a) a heavy chain variable region CDR1 comprising the amino acid sequence set forth in SEQ ID NO: 3, b) a heavy chain variable region CDR2 comprising the amino acid sequence set forth in SEQ ID NO: 4, c) a heavy chain variable region CDR3 comprising the amino acid sequence set forth in SEQ ID NO: 5, d) a light chain variable region CDR1 comprising the amino acid sequence set forth in SEQ ID NO: 6, e) a light chain variable region CDR2 comprising the amino acid sequence set forth in SEQ ID NO: 7, and f) a light chain variable region CDR3 comprising the amino acid sequence set forth in SEQ ID NO: 8.

In certain embodiments, the antibody or antigen-binding fragment thereof comprises: (a) a heavy chain variable region comprising the amino acid sequence set forth in SEQ ID NO: 1, and (b) a light chain variable region comprising the amino acid sequence set forth in SEQ ID NO: 2.

In certain embodiments, the radioactive isotope is ¹²⁴I, ¹³¹I, ¹⁷⁷Lu, or ⁹⁹mTc. In certain embodiments, the chelator compound is DOTA or DTPA

In certain embodiments, the antibody or antigen-binding fragment thereof comprises an amino acid sequence having at least about 80%, about 90%, about 95%, about 99% or about 100% homologous to the amino acid sequence set forth in SEQ ID NO: 17. In certain embodiments, the antibody or antigen-binding fragment thereof comprises the amino acid sequence set forth in SEQ ID NO: 17. In certain embodiments, the antibody or antigen-binding fragment thereof has amino acids 224-241 of SEQ ID NO: 17. In certain embodiments, the antibody or antigen-binding fragment thereof has amino acids 242-267 of SEQ ID NO: 17.

In certain embodiments, the therapeutic modality is a monoclonal antibody 3F8 (MoAb 3F8), a granulocyte-macrophage-colony-stimulating factor (GM-CSF), or a combination thereof. In certain embodiments, the therapeutic modality is administered into the CNS of the subject and/or systemically to the subject. In certain embodiments, wherein the therapeutic modality is administered to the subject concurrently or sequentially with the antibody or antigen-binding fragment thereof.

In certain embodiments, the antibody or antigen-binding fragment thereof is a DOTA-8H9 conjugate or a DTPA-8H9 conjugate. In certain embodiments, the antibody or antigen-binding fragment thereof is a ¹⁷⁷Lu-DOTA-8H9 conjugate or a ¹⁷⁷Lu-DTPA-8H9 conjugate or (177)LU-CHX-A″-DTPA-8H9.

In certain embodiments, the antibody or antigen-binding fragment thereof is a single chain variable fragment (scFv). In certain embodiments, the scFv comprises a portion of the amino acid sequence set forth in SEQ ID NO: 9, SEQ ID NO: 13, and SEQ ID NO: 14.

In another aspect, the present disclosure provides compositions comprising the antibody or antigen-binding fragment thereof disclosed herein.

In another aspect, the present disclosure provides pharmaceutical compositions comprising the antibody or antigen-binding fragment thereof disclosed herein, and a pharmaceutically acceptable carrier.

In another aspect, the present disclosure provides a method for imaging a tumor in a subject comprising administering to the subject an antibody or antigen-binding fragment thereof of disclosed herein.

In another aspect, the present disclosure provides an antibody or antigen-binding fragment thereof disclosed herein for use as a medicament.

In another aspect, the present disclosure provides an antibody or antigen-binding fragment thereof disclosed herein for use in the treatment of cancer.

In another aspect, the present disclosure provides an antibody or antigen-binding fragment thereof disclosed herein for use in the treatment of a central nerve system (CNS) cancer.

In another aspect, the present disclosure provides an antibody or antigen-binding fragment thereof disclosed herein for use in the treatment of metastatic CNS neuroblastoma, sarcoma, melanoma, ovarian carcinoma, and primary recurrent CNS malignancies.

In another aspect, the present disclosure provides an antibody or antigen-binding fragment thereof disclosed herein for use in a method for imaging a tumor in a subject. In another aspect, the present disclosure provides an antibody or antigen-binding fragment thereof disclosed herein for use in a method disclosed herein.

In another aspect, the present disclosure provides use of an antibody or antigen-binding fragment thereof disclosed herein for the preparation of a medicament for imaging tumor cells bearing the antigen recognized by the antibody or antigen-binding fragment thereof.

In another aspect, the present disclosure provides use of an antibody or antigen-binding fragment thereof disclosed herein for the preparation of a medicament for a method disclosed herein.

A. In certain non-limiting embodiments, the presently disclosed subject matter provides a method for treating a CNS cancer in an adult human subject, comprising administering into the CNS of the subject a therapeutically effective amount of an antibody or an antigen-binding fragment thereof that specifically binds to human B7H3, wherein the cancer is a primary central nerve system (CNS) cancer or a cancer metastatic to CNS, and the antibody or fragment is conjugated to a radioactive isotope and/or other therapeutic modality.

A1. The method of A, wherein the cancer is metastatic to leptomeninges.

A2. The method of A, wherein the cancer metastatic to CNS is a non-CNS solid tumor.

A3. The method of A2, wherein the solid tumor is selected from the group consisting of melanoma, ovarian cancer, and rhabdomyosarcoma.

A4. The method of A, wherein the antibody is selected from the group consisting of murine antibodies, humanized antibodies, chimeric antibodies, and human antibodies.

A5. The method of A, wherein the antibody is a murine antibody.

A6. The method of A, wherein the antibody or antigen-binding fragment thereof binds to FG-loop of B7H3.

A7. The method of A, wherein the antibody or antigen-binding fragment thereof comprises:
(a) a heavy chain variable region CDR1 comprising the amino acid sequence set forth in SEQ ID NO: 3 (NYDIN),
(b) a heavy chain variable region CDR2 comprising the amino acid sequence set forth in SEQ ID NO: 4 (WIFPGDGSTQY),
(c) a heavy chain variable region CDR3 comprising the amino acid sequence set forth in SEQ ID NO: 5 (QTTATWFAY),
(d) a light chain variable region CDR1 comprising the amino acid sequence set forth in SEQ ID NO: 6 (RASQSISDYLH),
(e) a light chain variable region CDR2 comprising the amino acid sequence set forth in SEQ ID NO: 7 (YASQSIS), and
(f) a light chain variable region CDR3 comprising the amino acid sequence set forth in SEQ ID NO: 8 (QNGHSFPLT).

A8. The method of A, wherein the antibody or antigen-binding fragment thereof comprises:
(a) a heavy chain variable region comprising the amino acid sequence set forth in SEQ ID NO: 1, and
(b) a light chain variable region comprising the amino acid sequence set forth in SEQ ID NO: 2.

A9. The method of A, wherein the antibody or antigen-binding fragment thereof is administered intrathecally to the subject.

A10. The method of A, wherein the antibody or antigen-binding fragment thereof is administered to the subject via an intraventricular device.

A11. The method of A, wherein the intraventricular device is an intraventricular catheter.

A12. The method of A, wherein the intraventricular device is an intraventricular reservoir.

A13. The method of A, the radioactive isotope is ¹³¹I, 177 Lu, or ^{99m}Tc.

A14. The method of A, comprising administering to the subject one treatment cycle of the antibody or antigen-binding fragment thereof.

A15. The method of A, comprising administering to the subject two treatment cycles of the antibody or antigen-binding fragment thereof.

A16. The method of A, wherein one treatment cycle comprises a dosimetry dose and a treatment dose.

A17. The method of A, wherein the therapeutically effective amount is about 10 mCi to about 200 mCi or about 10 mCI to about 100 mCi.

A18. The method of A, wherein the therapeutically effective amount is about 50 mCi.

A19. The method of A, wherein the method prolongs survival of the subject.

A20. The method of A, wherein the antibody or antigen-binding fragment thereof binds to a human B7H3 polypeptide comprising an amino acid sequence having at least about 80%, about 90%, about 95%, about 99% or about 100% homologous to the amino acid sequence set forth in SEQ ID NO: 17.

A21. The method of A, wherein the antibody or antigen-binding fragment thereof binds to a human B7H3 polypeptide comprising the amino acid sequence set forth in SEQ ID NO: 17.

A22. The method of A, wherein the antibody or antigen-binding fragment thereof binds to a human B7H3 polypeptide having amino acids 224-241 of SEQ ID NO: 17.

A23. The method of A, wherein the antibody or antigen-binding fragment thereof binds to a human B7H3 polypeptide having amino acids 242-267 of SEQ ID NO: 17.

A24. The method of A, further comprising administering to the subject an additional therapeutic modality, for example, but not limited to, one or more chemotherapeutic agent, one or more checkpoint inhibitor agent, and/or radiation therapy. Such one or more additional therapeutic may be administered into the CNS and/or systemically, either concurrently or sequentially with the B7H3-directed antibodies or antigen-binding fragments described herein.

### DETAILED DESCRIPTION OF THE INVENTION

All publications, patents and other references cited herein are incorporated by reference in their entirety into the present disclosure.

For purposes of clarity of disclosure and not by way of limitation, the detailed description is divided into the following subsections:

1. Definitions

2. Anti-B7H3 antibodies

3. Methods of treatment

### 1. Definitions

In the description that follows, certain conventions will be followed as regards the usage of terminology. Generally, terms used herein are intended to be interpreted consistently with the meaning of those terms as they are known to those of skill in the art.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein interchangeably, the terms “antigen-binding portion”, “antigen-binding fragment”, or “antigen-binding region” of an antibody, refer to the region or portion of an antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U.S. Department of Health and Human Services, National Institutes of Health (1987). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of ordinary skill in the art, and the fragments are screened for utility in the same manner as are intact antibodies.

As used herein, an antibody that “specifically binds to B7H3” refers to an antibody that binds to B7H3 (e.g., human B7H3) with a K_{d} of 5×10⁻⁷M or less, 1×10⁻⁷ M or less, 5×10⁻⁸M or less, 1×10⁻⁸ M or less, 5×10⁻⁹M or less, 1×10⁻⁹M or less, 5×10⁻¹⁰ M or less, 1×10⁻¹⁰ M or less, 5×10⁻¹¹M or less or 1×10⁻¹¹ M or less.

An “antibody that competes for binding” or “antibody that cross-competes for binding” with a reference antibody for binding to an antigen, e.g., B7H3, refers to an antibody that blocks binding of the reference antibody to the antigen (e.g., B7H3) in a competition assay by about 50% or more, e.g., about 55% or more, about 60% or more, about 65% or more, about 70% or more, about 75% or more, about 80% or more, about 85% or more, about 90% or more, about 95% or more, about 98% or more or about 99% or more, and conversely, the reference antibody blocks binding of the antibody to the antigen (e.g., B7H3) in a competition assay by about 50% or more, e.g., about 55% or more, about 60% or more, about 65% or more, about 70% or more, about 75% or more, about 80% or more, about 85% or more, about 90% or more, about 95% or more, about 98% or more or about 99% or more. An exemplary competition assay is described in “Antibodies,” Harlow and Lane (Cold Spring Harbor Press, Cold Spring Harbor, N.Y.) (1988). In certain embodiments, the reference antibody is a murine anti-B7H3 antibody. In certain embodiments, the reference antibody is 8H9.

An “antibody or antigen-binding fragment that competes for binding” or “antibody or antigen-binding fragment that cross-competes for binding” with a reference antibody for binding to an antigen, e.g., B7H3, refers to an antibody or an antigen-binding fragment that blocks binding of the reference antibody to the antigen (e.g., B7H3) in a competition assay by about 50% or more, e.g., about 55% or more, about 60% or more, about 65% or more, about 70% or more, about 75% or more, about 80% or more, about 85% or more, about 90% or more, about 95% or more, about 98% or more or about 99% or more, and conversely, the reference antibody blocks binding of the antibody or antigen-binding fragment to the antigen (e.g., B7H3) in a competition assay by about 50% or more, e.g., about 55% or more, about 60% or more, about 65% or more, about 70% or more, about 75% or more, about 80% or more, about 85% or more, about 90% or more, about 95% or more, about 98% or more or about 99% or more. An exemplary competition assay is described in “Antibodies,” Harlow and Lane (Cold Spring Harbor Press, Cold Spring Harbor, N.Y.) (1988).

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and ^{e-100} indicating a closely related sequence.

A “therapeutically effective amount” of an agent, e.g., an anti-B7H3 antibody or an antigen-binding fragment thereof, refers to an amount effective, at dosages and for periods of time necessary, to achieve the desired therapeutic or prophylactic result, e.g., treating a cancer (e.g., primary cancers to CNS or cancers metastatic to CNS, e.g., leptomeninges).

A “subject”, as referred to herein, may be a human or non-human subject, such as, but not limited to, a non-human primate, a dog, a cat, a horse, a rodent, a rabbit, etc. An adult human subject is a subject that has attained an age of at least 18 years or at least 20 years. An adult non-human subject is a subject that has attained sexual maturity. A human subject that is not an adult is a pediatric subject.

“Administering into the CNS of the subject”, as used herein, means administering into one or more of the cerebrospinal fluid, subarachnoid space, meningeal tissue, and/or nervous system (brain and/or spinal cord) tissue of the subject.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, prolonging survival, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a cancer primary to CNS or a cancer metastatic to CNS (e.g., leptomeninges).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

### 2. Anti-B7H3 Antibodies

The presently disclosed subject matter provides uses of anti-B7H3 antibodies or antigen-binding fragments thereof for treating cancers, e.g., cancers primary to CNS or cancers metastatic to CNS (e.g., to the parenchyma or to the leptomeninges). The anti-B7H3 antibodies can be murine, humanized, chimeric, or human antibodies.

In certain embodiments, the anti-B7H3 antibodies or antigen-binding fragments thereof bind to a B7H3 polypeptide. In certain embodiments, the B7H3 polypeptide is a human B7H3 polypeptide. The B7H3 polypeptide can have an amino acid sequence that is at least about 10%, about 20%, about 30%, about 40%, about 50%, about 60%, about 70%, about 80%, about 90%, about 95%, about 99%, or about 100% homologous to SEQ ID NO: 17 (homology herein may be determined using standard software such as BLAST or FASTA) as provided below, or fragments thereof, and/or may optionally comprise up to one or up to two or up to three amino acid substitutions (e.g., conservative substitutions). In certain embodiments, the B7H3 polypeptide comprises the amino acid sequence set forth in SEQ ID NO: 17. In certain embodiments, the B7H3 polypeptide can have an amino acid sequence that is a consecutive portion of SEQ ID NO: 17 which is at least 10, at least 15, at least 20, at least 25, at least 30, at least 40, at least 50, at least 60, at least 70, at least 80, at least 90, at least 100, at least 150, at least 200, at least 250, at least 300, at least 350, at least 400, at least 450, at least 500, and up to 534 amino acids in length. Alternatively or additionally, in non-limiting various embodiments, the B7H3 polypeptide has an amino acid sequence of amino acids 1 to 534, 1 to 50, 50 to 100, 100 to 150, 150 to 200, 200 to 250, 224 to 241, 242 to 267, 241 to 267, 250 to 300, 300 to 350, or 350 to 400, 400 to 450, 450 to 500, and 500 to 534 of SEQ ID NO: 17. In certain embodiments, the B7H3 polypeptide comprises amino acids 224 to 241 of SEQ ID NO: 17. Amino acids 224-241 of SEQ ID NO: 17 has the amino acid sequence of NPVLQQDAHSSVTITPQR (SEQ ID NO: 15). In certain embodiments, the B7H3 polypeptide comprises amino acids 242 to 267 of SEQ ID NO: 17. Amino acids 242 to 267 of SEQ ID NO: 17 has the amino acid sequence of SPTGAVEVQVPEDPVVALVGTDATLR (SEQ ID NO: 16).

(SEQ ID NO: 17)
MLRRRGSPGMGVHVGAALGALWFCLTGALEVQVPEDPVVALVGTDATLCCS
FSPEPGFSLAQLNLIWQLTDTKQLVHSFAEGQDQGSAYANRTALFPDLLAQ
GNASLRLQRVRVADEGSFTCFVSIRDFGSAAVSLQVAAPYSKPSMTLEPNK
DLRPGDTVTITCSSYQGYPEAEVFWQDGQGVPLTGNVTTSQMANEQGLFDV
HSILRVVLGANGTYSCLVRNPVLQQDAHSSVTITPQRSPTGAVEVQVPEDP
VVALVGTDATLRCSFSPEPGFSLAQLNLIWQLTDTKQLVHSFTEGRDQGSA
YANRTALFPDLLAQGNASLRLQRVRVADEGSFTCFVSIRDFGSAAVSLQVA
APYSKPSMTLEPNKDLRPGDTVTITCSSYRGYPEAEVFWQDGQGVPLTGNV
TTSQMANEQGLFDVHSVLRVVLGANGTYSCLVRNPVLQQDAHGSVTITGQP
MTFPPEALWVTVGLSVCLIALLVALAFVCWRKIKQSCEEENAGAEDQDGEG
EGSKTALQPLKHSDSKEDDGQEIA

In certain embodiments, the anti-B7H3 antibody is a murine antibody. In certain embodiments, the anti-B7H3 antibody is antibody 8H9, which is disclosed in U.S. Pat. Nos. 7,737,258, 7,666,424, 8,148,154, 7,740,845, 8,414,892, 9,062,110, and 8,501,471, and International Patent Publication No. WO2008/116219, all of which are incorporated by reference in their entireties.

In certain embodiments, the anti-B7H3 antibody or antigen-binding fragment thereof comprises a heavy chain variable region CDR1 comprising the amino acid sequence set forth in SEQ ID NO: 3 (NYDIN), a heavy chain variable region CDR2 comprising the amino acid sequence set forth in SEQ ID NO: 4 (WIFPGDGSTQY), a heavy chain variable region CDR3 comprising the amino acid sequence set forth in SEQ ID NO: 5 (QTTATWFAY), a light chain variable region CDR1 comprising the amino acid sequence set forth in SEQ ID NO: 6 (RASQSISDYLH), a light chain variable region CDR2 comprising the amino acid sequence set forth in SEQ ID NO: 7 (YASQSIS), and/or a light chain variable region CDR3 comprising the amino acid sequence set forth in SEQ ID NO: 8 (QNGHSFPLT). In certain embodiments, the anti-B7H3 antibody comprises (a) a heavy chain variable region comprising the amino acid sequence set forth in SEQ ID NO: 1, and/or (b) a light chain variable region comprising the amino acid sequence set forth in SEQ ID NO: 2. SEQ ID NOs: 1-8 are provided below.

(SEQ ID NO: 1)
QVQLQQSGAELVKPGASVKLSCKASGYTFTNYDINWVRQRPEQGLEWIGWI
FPGDGSTQYNEKFKGKATLTTDTSSSTAYMQLSRLTSEDSAVYFCARQTTA
TWFAYWGQGTLVTVSAAKTTPPSVYPLAPGSAAQTNSMVTLGCLVKGYFPE
PVTVTWNSGSLSSGVHTFPAVLQSDLYTLSSSVTVPSSTWPSETVTCNVAH
PASSTKVDKKIVPRDCGCKPCICTVPEVSSVFIFPPKPKDVLTITLTPKVT
CVVVDISKDDPEVQFSWFVDDVEVHTAQTQPREEQFNSTERSVSELPIMHQ
DWLNGKEFKCRVNSAAFPAPIEKTISKTKGRPKAPQVYTIPPPKEQMAKDK
VSLTCMITDFFPEDITVEWQWNGQPAENYKNTQPIMDTDGSYFVYSKLNVQ
KSNWEAGNTFTCSVLHEGLHNHHTEKSLSHSPGK
(SEQ ID NO: 2)
DIVMTQSPATLSVTPGDRVSLSCRASQSISDYLHWYQQKSHESPRLLIKYA
SQSISGIPSRFSGSGSGSDFTLSINSVEPEDVGVYYCQNGHSFPLTFGAGT
KLELKRADAAPTVSIFPPSSEQLTSGGASVVCFLNNFYPKDINVKWKIDGS
ERQNGVLNSWTDQDSKDSTYSMSSTLTLTKDEYERHNSYTCEATHKTSTSP
IVKSFNRNEC
(SEQ ID NO: 3)
NYDIN
(SEQ ID NO: 4)
WIFPGDGSTQY
(SEQ ID NO: 5)
QTTATWFAY
(SEQ ID NO: 6)
RASQSISDYLH
(SEQ ID NO: 7)
YASQSIS
(SEQ ID NO: 8)
QNGHSFPLT

In certain embodiments, the anti-B7H3 antibody or antigen-binding fragment thereof cross-competes for binding to B7H3 with antibody 8H9. In certain embodiments, the anti-B7H3 antibody or antigen-binding fragment thereof cross-competes for binding to B7H3 with a reference antibody that comprises a heavy chain variable region CDR1 comprising the amino acid sequence set forth in SEQ ID NO: 3 (NYDIN), a heavy chain variable region CDR2 comprising the amino acid sequence set forth in SEQ ID NO: 4 (WIFPGDGSTQY), a heavy chain variable region CDR3 comprising the amino acid sequence set forth in SEQ ID NO: 5 (QTTATWFAY), a light chain variable region CDR1 comprising the amino acid sequence set forth in SEQ ID NO: 6 (RASQSISDYLH), a light chain variable region CDR2 comprising the amino acid sequence set forth in SEQ ID NO: 7 (YASQSIS), and a light chain variable region CDR3 comprising the amino acid sequence set forth in SEQ ID NO: 8 (QNGHSFPLT). In certain embodiments, the anti-B7H3 antibody or antigen-binding fragment thereof cross-competes for binding to B7H3 with a reference antibody that comprises (a) a heavy chain variable region comprising the amino acid sequence set forth in SEQ ID NO: 1, and (b) a light chain variable region comprising the amino acid sequence set forth in SEQ ID NO: 2.

In certain embodiments, the anti-B7H3 antibody or antigen-binding fragment thereof binds to the same epitope on B7H3 as antibody 8H9. In certain embodiments, the anti-B7H3 antibody or antigen-binding fragment thereof binds to the same epitope on B7H3 as a reference antibody that comprises a heavy chain variable region CDR1 comprising the amino acid sequence set forth in SEQ ID NO: 3 (NYDIN), a heavy chain variable region CDR2 comprising the amino acid sequence set forth in SEQ ID NO: 4 (WIFPGDGSTQY), a heavy chain variable region CDR3 comprising the amino acid sequence set forth in SEQ ID NO: 5 (QTTATWFAY), a light chain variable region CDR1 comprising the amino acid sequence set forth in SEQ ID NO: 6 (RASQSISDYLH), a light chain variable region CDR2 comprising the amino acid sequence set forth in SEQ ID NO: 7 (YASQSIS), and a light chain variable region CDR3 comprising the amino acid sequence set forth in SEQ ID NO: 8 (QNGHSFPLT). In certain embodiments, the anti-B7H3 antibody or antigen-binding fragment thereof binds to the same epitope on B7H3 as a reference antibody that comprises (a) a heavy chain variable region comprising the amino acid sequence set forth in SEQ ID NO: 1, and (b) a light chain variable region comprising the amino acid sequence set forth in SEQ ID NO: 2.

In certain embodiments, the anti-B7H3 antibody is a single chain variable fragment (scFv). The scFv can be a murine, humanized or human scFv. In certain embodiments, the anti-B7H3 antibody is a murine scFv. In certain embodiments, the anti-B7H3 antibody is a scFv comprising the amino acid sequence set forth in SEQ ID NO: 9 (provided below). In certain embodiments, the anti-B7H3 antibody is a scFv comprising the amino acid sequence set forth in SEQ ID NO: 13 (provided below). In certain embodiments, the anti-B7H3 antibody is a scFv comprising the amino acid sequence set forth in SEQ ID NO: 14 (provided below).

(SEQ ID NO: 9)
QVKLQQSGAELVKPGASVKLSCKASGYTFTNYDINWVRQRPEQGLEWIGWI
FPGDGSTQYNEKFKGKATLTTDTSSSTAYMQLSRLTSEDSAVYFCARQTTA
TWFAYWGQGTTVTVSSGGGGSGGGGSGGGGSDIELTQSPTTLSVTPGDRVS
LSCRASQSISDYLHWYQQKSHESPRLLIKYASQSISGIPSRFSGSGSGSDF
TLSINSVEPEDVGVYYCQNGHSFPLTFGAGTKLELKQAA
(SEQ ID NO: 13)
QVKLQQSGAELVKPGASVKLSCKASGYTFTNYDINWVRQRPEQGLEWIGWI
FPGDGSTQYNEKFKGKATLTTDTSSSTAYMQLSRLTSEDSAVYFCARQTTA
TWFAYWGQGTTVTVSSDGGGSGGGGSGGGGSDIELTQSPTTLSVTPGDRVS
LSCRASQSISDYLHWYQQKSHESPRLLIKYASQSISGIPSRFSGSGSGSDF
TLSINSVEPEDVGVYYCQNGHSFPLTFGAGTKLELKQAA
(SEQ ID NO: 14)
QVKLQQSGAELVEPGASVKLSCKASGYTFTNYDINWVRQRPEQGLEWIGWI
FPGDGSTQYNEKFKGKATLTTDTSSSTAYMQLSRLTSEDSAVYFCARQTTA
TWFAYWGQGTTVTVSSDGGGSGGGGSGGGGSDIELTQSPTTLSVTPGDQVS
LSCRASQSISDYLHWYQQKSHESPQLLIKYASQSISGIPSRFSGSGSGSDF
TLSINSVEPEDVGVYYCQNGHSFPLTFGAGTELELEQAA

The nucleotide sequence encoding SEQ ID NO: 9 is SEQ ID NO: 10 (sense) and SEQ ID NO: 11 (complementary). SEQ ID NOs: 10 and 11 are provided below.

(SEQ ID NO: 10)
caggtcaaac tgcagcagtc tggggctgaa ctggtaaagc
ctggggcttc agtgaaattg tcctgcaagg cttctggcta
caccttcaca aactatgata taaactgggt gaggcagagg
cctgaacagg gacttgagtg gattggatgg atttttcctg
gagatggtag tactcaatac aatgagaagt tcaagggcaa
ggccacactg actacagaca catcctccag cacagcctac
atgcagctca gcaggctgac atctgaggac tctgctgtct
atttctgtgc aagacagact acggctacct ggtttgctta
ctggggccaa gggaccacgg tcaccgtctc ctcaggtgga
ggcggttcag gcggaggtgg ctctggcggt ggcggatcgg
acatcgagct cactcagtct ccaaccaccc tgtctgtgac
tccaggagat agagtctctc tttcctgcag ggccagccag
agtattagcg actacttaca ctggtaccaa caaaaatcac
atgagtctcc aaggcttctc atcaaatatg cttcccaatc
catctctggg atcccctcca ggttcagtgg cagtggatca
gggtcagatt tcactctcag tatcaacagt gtggaacctg
aagatgttgg agtgtattac tgtcaaaatg gtcacagctt
tccgctcacg ttcggtgctg ggaccaagct ggagctgaaa
caggcggccg c
(SEQ ID NO: 11)
gtccagtttg acgtcgtcag accccgactt gaccatttcg
gaccccgaag tcactttaac aggacgttcc gaagaccgat
gtggaagtgt ttgatactat atttgaccca ctccgtctcc
ggacttgtcc ctgaactcac ctaacctacc taaaaaggac
ctctaccatc atgagttatg ttactcttca agttcccgtt
ccggtgtgac tgatgtctgt gtaggaggtc gtgtcggatg
tacgtcgagt cgtccgactg tagactcctg agacgacaga
taaagacacg ttctgtctga tgccgatgga ccaaacgaat
gaccccggtt ccctggtgcc agtggcagag gagtccacct
ccgccaagtc cgcctccacc gagaccgcca ccgcctagcc
tgtagctcga gtgagtcaga ggttggtggg acagacactg
aggtcctcta tctcagagag aaaggacgtc ccggtcggtc
tcataatcgc tgatgaatgt gaccatggtt gtttttagtg
tactcagagg ttccgaagag tagtttatac gaagggttag
gtagagaccc taggggaggt ccaagtcacc gtcacctagt
cccagtctaa agtgagagtc atagttgtca caccttggac
ttctacaacc tcacataatg acagttttac cagtgtcgaa
aggcgagtgc aagccacgac cctggttcga cctcgacttt
gtccgccggc g

In certain embodiments, the nucleotide sequence encoding a scFv that binds to a B7H3 polypeptide has the nucleotide sequence set forth in SEQ ID NO: 12 (provided below).

(SEQ ID NO: 12)
caggtcaaac tgcagcagtc tggggctgaa ctggtaaagc
ctggggcttc agtgaaattg tcctgcaagg cttctggcta
caccttcaca aactatgata taaactgggt gaggcagagg
cctgaacagg gacttgagtg gattggatgg atttttcctg
gagatggtag tactcaatac aatgagaagt tcaagggcaa
ggccacactg actacagaca catcctccag cacagcctac
atgcagctca gcaggctgac atctgaggac tctgctgtct
atttctgtgc aagacagact acggctacct ggtttgctta
ctggggccaa gggaccacgg tcaccgtctc ctcagatgga
ggcggttcag gcggaggtgg ctctggcggt ggcggatcgg
acatcgagct cactcagtct ccaaccaccc tgtctgtgac
tccaggagat agagtctctc tttcctgcag ggccagccag
agtattagcg actacttaca ctggtaccaa caaaaatcac
atgagtctcc aaggcttctc atcaaatatg cttcccaatc
catctctggg atcccctcca ggttcagtgg cagtggatca
gggtcagatt tcactctcag tatcaacagt gtggaacctg
aagatgttgg agtgtattac tgtcaaaatg gtcacagctt
tccgctcacg ttcggtgctg ggaccaagct ggagctgaaa
caggcggccg c

In certain embodiments, the anti-B7H3 antibody is a humanized antibody. In certain embodiments, the anti-B7H3 antibody is a humanized anti-B7H3 antibody disclosed in International Patent Publication No. WO2016/033225, which is incorporated by reference in its entirety. In certain embodiments, the anti-B7H3 antibody is a humanized B7-H3-reactive antibody disclosed in U.S. Pat. Nos. 8,802,091 and 9,441,049, both of which are incorporated by reference in their entireties.

In certain embodiments, the anti-B7H3 antibody or antigen-binding fragment thereof cross-competes for binding to B7H3 with a humanized anti-B7H3 antibody disclosed in International Patent Publication No. WO2016/033225. In certain embodiments, the anti-B7H3 antibody or antigen-binding fragment thereof binds to the same epitope on B7H3 as a humanized anti-B7H3 antibody disclosed in International Patent Publication No. WO2016/033225.

In certain embodiments, the anti-B7H3 antibody or antigen-binding fragment thereof cross-competes for binding to B7H3 with a humanized B7-H3-reactive antibody disclosed in U.S. Pat. Nos. 8,802,091 and 9,441,049. In certain embodiments, the anti-B7H3 antibody or antigen-binding fragment thereof binds to the same epitope on B7H3 as a humanized B7-H3-reactive antibody disclosed in U.S. Pat. Nos. 8,802,091 and 9,441,049.

For example, and not by way of limitation, the cross-competing antibodies can bind to the same epitope region, e.g., same epitope, adjacent epitope or overlapping epitope as a reference antibody (e.g., antibody 8H9, a humanized anti-B7H3 antibody disclosed in International Patent Publication No. WO2016/033225, or a humanized B7-H3-reactive antibody disclosed in U.S. Pat. Nos. 8,802,091 and 9,441,049).

Such cross-competing antibodies can be identified based on their ability to cross-compete with the reference antibody in standard B7H3 binding assays. For example, Biacore analysis, ELISA assays or flow cytometry can be used to demonstrate cross-competition with the reference antibody. The ability of a test antibody to inhibit the binding of a reference antibody to B7H3 (e.g., human B7H3) demonstrates that the test antibody can compete with the reference antibody for binding to B7H3, and thus binds to the same epitope region on B7H3 as the reference antibody. In certain embodiments, the cross-competing antibody binds to the same epitope on B7H3 (e.g., human B7H3) as the reference antibody.

In a non-limiting example of a competition assay, immobilized antigen, e.g., a B7H3 (e.g., human B7H3) polypeptide, can be incubated in a solution comprising a first labeled antibody that binds to the antigen and a second unlabeled antibody that is being tested for its ability to compete with the first antibody for binding to the antigen. In certain embodiments, the second antibody can be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced, e.g., greater than about 50%, in the test sample relative to the control sample, then that indicates that the second antibody is competing with the first antibody for binding to the antigen. See Harlow and Lane (1988) Antibodies: A Laboratory Manual ch. 14 (Cold Spring Harbor Laboratory, Cold Spring Harbor, N.Y.).

In certain embodiments, the cross-competing antibody has a K_{d} of about 5×10⁻⁷ M or less, about 1×10⁻⁷ M or less, about 5×10⁻⁸ M or less, about 1×10⁻⁸ M or less, about 5×10⁻⁹ M or less, about 1×10⁻⁹ M or less, about 5×10⁻¹⁰ M or less, or about 1×10⁻¹⁰ M or less, to B7H3 (e.g., human B7H3).

In certain embodiments, the anti-B7H3 antibody is conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, ²¹¹At, ¹⁴C, ⁵¹Cr, ⁵⁷Co, ⁵⁸Co, ⁶⁷Cu, ¹⁵²Eu, ⁶⁷Ga, ³H, ¹¹¹In, ⁵⁹Fe, ²¹²Pb, ¹⁷⁷Lu, ³²P, ²²³Ra, ²²⁴Ra, ¹⁸⁶Re, ¹⁸⁸Re, ⁷⁵Se, ³⁵S, ^{99m}Tc, ²²⁷Th, ⁸⁹Zr, ⁹⁰Y, ¹²³I, ¹²⁴I, ¹²⁵I, ¹³¹I, and alpha-emitting particles. Non-limiting examples of alpha-emitting particles include ²⁰⁹Bi, ²¹¹Bi, ²¹²Bi, ²¹³Bi, ²¹⁰Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Rd, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pn, ²³⁹Pn, ²⁴⁰Pn, ²⁴⁴Pn, ²⁴¹Am, ²⁴⁴Cn, ²⁴⁵Cn, ²⁴⁸Cn, ²⁴⁹Cf, and ²⁵²Cf. In certain embodiments the radioactive isotopes may be selected among ^{94m}Tc, ⁶⁴Cu, ⁶⁸Ga, ⁶⁶Ga, ⁷⁶Br, ⁸⁶Y, ⁸²Rb, ^{110m}In, ¹³N, ¹¹C and ¹⁸F. Methods for preparing radioimmunoconjugates are established in the art. Examples of radioimmunoconjugates are commercially available, including Zevalin™ (DEC Pharmaceuticals) and Bexxar™ (Corixa Pharmaceuticals), and similar methods can be used to prepare radioimmunoconjugates using the antibodies of the invention.

Radioactively labeled antibody agents may be produced according to well-known technologies in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Provided antibody agents may be labeled with technetium-99m by ligand exchange process, for example, by reducing pertechnate with stannous solution, chelating the reduced technetium onto a Sephadex column and applying the antibody to this column. In certain embodiments, provided antibody agents are labeled using direct labeling techniques, e.g., by incubating pertechnate, a reducing agent such as SNCI2, a buffer solution such as sodium-potassium phthalate solution, and the antibody. Intermediary functional groups which are often used to bind radioisotopes which exist as metallic ions to antibody are diethylenetriaminepentaacetic acid (DTPA), or ethylene diaminetetracetic acid (EDTA), or 1,4,7,10-tetraazacyclododecane-1,4,7,10-tetraacetic acid (DOTA), or p-aminobenzyl-DOTA (DOTA-Bn). Radioactive isotopes may be detected by, for example, dosimetry.

1,4,7,10-tetraazacyclododecane-1,4,7,10-tetraacetic acid (also known as DOTA) is an organic compound with the formula (CH₂CH₂NCH₂CO₂H)₄. The molecule consists of a central 12-membered tetraaza (i.e., containing four nitrogen atoms) ring. DOTA is used as a complexing agent, especially for lanthanide ions. Its complexes have medical applications as contrast agents and cancer treatments. There are several forms of DOTA, each having different kinetics and stability constants. The bifunctional chelating agents can bind metals and still possess a chemically reactive functional group for covalent attachment to peptides.

DOTA can be conjugated to monoclonal antibodies by attachment of one of the four carboxyl groups as an amide.

Pentetic acid or diethylenetriaminepentaacetic acid (DTPA) is an aminopolycarboxylic acid consisting of a diethylenetriamine backbone with five carboxymethyl groups. DTPA has the molecular formula C₁₄H₂₃N₃O₁₀ and the IUPAC name 2-[bis[2-[bis(carboxymethyl)amino]ethyl]amino]acetic acid. DTPA is an edetate and a chelating agent used in preparing radiopharmaceuticals. DTPA may chelate metallic moieties of unbound, extracellular radioimmunotherapeutics, thereby aggregating radioimmunotherapeutics locally to higher concentrations, and improving tumor cell radiocytotoxicity, while sparing normal tissues from the radiocytotoxic effects. In addition, DTPA is used in radioimaging procedures as complexes with radioisotopes. As a chelating agent, DTPA wraps around a metal ion by forming up to eight bonds. Transition metals usually form less than eight coordination bonds, so DTPA still has the ability to bind to other reagents after forming a complex with these metals.

In certain embodiments, the number of DOTA molecules or DTPA molecules used for conjugation per antibody agent is selected from the group consisting of 1, 2, 3, 4, 5, 6, 7, 8, 9, or 10 DOTA molecules or DTPA molecules. In certain embodiments, DOTA or DTPA is an intermediary functional group. In certain embodiments, the radioactively labeled antibody comprises 2 or 3 DOTA molecules or DTPA molecules.

High specific activities used with DOTA-peptides radiolabeled with ⁹⁰Y, ⁶⁸Ga, and ¹⁷⁷Lu are achievable. Even higher specific activities can be achieved when radiolabeling DTPA-peptides. A major drawback of using DOTA-peptides is that the incorporation of the radiometal requires heating and time, while DTPA-peptides can be radiolabeled at room temperature. While a certain amount of radioactivity is necessary for the detection of target tissue uptakes by imaging systems, concordant low mass doses of DTPA-peptides can be administered.

In certain embodiments the radioactively labeled antibody agent is a ¹⁷⁷Lu-DOTA-8H9 conjugate or a ¹⁷⁷Lu-DTPA-8H9 conjugate or (177)LU-CHX-A″-DTPA-8H9.

In certain embodiments a pediatric human subject is treated for a cancer, including but not limited to a cancer primary or metastatic to the CNS, by administration of a therapeutically effective amount of ¹⁷⁷Lu-DOTA-8H9, ¹⁷⁷Lu-DTPA-8H9 or (177)LU-CHX-A″-DTPA-8H9. In certain related embodiments, the ¹⁷⁷Lu-DOTA-8H9, ¹⁷⁷Lu-DTPA-8H9 or (177)LU-CHX-A″-DTPA-8H9 is administered into the CNS of the pediatric human subject.

### 3. Methods of Treatment

Anti-B7H3 antibodies of the presently disclosed subject matter can be administered for therapeutic treatments to a human subject (e.g., an adult human subject) suffering from a cancer (e.g., a cancer primary to CNS, or a cancer metastatic to CNS) in an amount sufficient to prevent, inhibit or reduce the progression of the cancer. Progression includes, e.g., the growth, invasiveness, metastases and/or recurrence of the cancer.

In certain embodiments, the anti-B7H3 antibodies or antigen-binding fragments thereof prolong survival of the subject relative to a control subject or control subject population not receiving said treatment. In certain embodiments, the period of survival is extended at least about 25 percent, or at least about 30 percent, or at least about 50 percent.

In certain embodiments, the anti-B7H3 antibodies or antigen-binding fragments thereof prolong the remission of the cancer in the subject relative to a control subject or control subject population not receiving said treatment.

In certain embodiments, the method includes administering to the subject a therapeutically effective amount of an anti-B7H3 antibody or an antigen-binding fragment thereof (or a pharmaceutical composition thereof) to produce an anti-cancer effect in the subject. In certain embodiments, an “anti-cancer effect” means one or more of: a reduction in aggregate cancer cell mass, a reduction in cancer cell growth rate, a reduction in cancer cell proliferation, a reduction in tumor mass, a reduction in tumor volume, a reduction in cancer cell proliferation, a reduction in cancer growth rate or a reduction in cancer metastasis. In certain embodiments, the anti-cancer effect is a reduction in the number of cancer cells. In certain embodiments, where the cancer is a solid tumor, an anti-cancer effect can be a reduction in tumor size and/or a reduction in the rate of tumor growth. In certain embodiments, the anti-cancer effect is a reduction of NB cells in the cerebrospinal fluid. In certain embodiments, the anti-cancer effect is a reduction in the aggregate cancer cell burden. In certain embodiments, the anti-cancer effect is a reduction in the rate of cell proliferation and/or an increase in the rate of cell death. In certain embodiments, the anti-cancer effect is a prolongation of survival. In certain embodiments, the anti-cancer effect is a prolongation in the interval until relapse relative to a control subject or control subject population not receiving said treatment.

A therapeutically effective amount can depend upon the severity of the disease and the general state of the subject's own immune system. In certain embodiments, the anti-B7H3 antibody or antigen-binding fragment thereof is conjugated to a radioactive isotope, e.g., those disclosed herein (e.g., ¹³¹I). In certain embodiments, the therapeutically effective amount is from about 1 mCi to about 200 mCi, e.g., from about 1 mCi to about 10 mCi, from about 10 mCi to about 200 mCi, from about 10 mCi to about 160 mCi, from about 10 mCi to about 120 mCi, from about 10 mCi to about 100 mCi, from about 10 mCi to about 70 mCi, from about 10 mCi to about 50 mCi, from about 50 mCi to about 200 mCi, from about 100 to about 200 mCi, from about 100 mCi to about 120 mCi, from about 120 mCi to about 160 mCi, from about 50 mCi to about 100 mCi, from about 40 mCi to about 60 mCi, from about 60 mCi to about 80 mCi, or from about 80 mCi to about 100 mCi. In certain embodiments, the therapeutically effective amount is from about 40 mCi to about 60 mCi. In certain embodiments, the therapeutically effective amount is about 10 mCi, about 20 mCi, about 30 mCi, about 40 mCi, about 50 mCi, about 60 mCi, about 70 mCi, about 80 mCi, about 90 mCi, about 100 mCi, about 110 mCi, about 120 mCi, about 130 mCi, about 140 mCi, about 150 mCi, about 160 mCi, about 170 mCi, about 180 mCi, about 190 mCi, or about 200 mCi. In certain embodiments, the therapeutically effective amount is about 50 mCi. In certain embodiments, the therapeutically effective amount is no greater than about 100 mCi. In certain embodiments, the therapeutically effective amount is no greater than about 50 mCi.

In certain embodiments, the method comprises administering one treatment cycle of the anti-B7H3 antibody or antigen-binding fragment thereof to the subject. In certain embodiments, the method comprises administering up to two, up to three, up to four, or up to five treatment cycles of the anti-B7H3 antibody or antigen-binding fragment thereof to the subject. In certain embodiments, the method comprises administering two treatment cycles of the anti-B7H3 antibody or antigen-binding fragment thereof to the subject. In certain embodiments, the method comprises administering additional treatment cycles (e.g., two new treatment cycles) to a relapsed patient.

In certain embodiments, one treatment cycle comprises a treatment dose. In certain embodiments, the treatment dose is from about 1 mCi to about 100 mCi, e.g., from about 1 mCi to about 10 mCi, from about 10 mCi to about 100 mCi, from about 10 mCi to about 40 mCi, from about 10 mCi to about 70 mCi, from about 10 mCi to about 50 mCi, from about 50 mCi to about 100 mCi, from about 40 mCi to about 60 mCi, from about 60 mCi to about 80 mCi, or from about 80 mCi to about 100 mCi. In certain embodiments, the treatment dose is from about 40 mCi to about 60 mCi. In certain embodiments, the treatment dose is about 50 mCi. In certain embodiments, the treatment dose is administered during week 1 of one treatment cycle. In certain embodiments, the treatment dose is administered during week 2 of one treatment cycle.

In certain embodiments, one treatment cycle comprises a dosimetry dose and a treatment dose. In certain embodiments, the dosimetry dose is from about 1 mCi to about 10 mCi, e.g., from about 1 mCi to about 3 mCi, from about 3 mCi to about 5 mCi, from about 5 mCi to about 7 mCi, or from about 7 mCi to about 10 mCi). In certain embodiments, the dosimetry dose is about 1 mCi, about 2 mCi, about 3 mCi, about 4 mCi, about 5 mCi, about 6 mCi, about 7 mCi, about 8 mCi, about 9 mCi or about 10 mCi. In certain embodiments, the dosimetry dose is about 2 mCi. In certain embodiments, the dosimetry dose is administered prior to the treatment dose. In certain embodiments, the dosimetry dose is administered during week 1 of one treatment cycle.

In certain embodiments, one treatment cycle further comprises an observation period. In certain embodiments, the observation period lasts for about 2 weeks, and follows the treatment dose. In certain embodiments, one treatment cycle further comprises post-treatment evaluations. In certain embodiments, the post-treatment evaluations last for about 1 week, and follows the observation period.

In certain embodiments, the treatment is administered after the subject has been treated with one or more other cancer treatments. In certain embodiments, the above treatment is administered simultaneously or sequentially while the subject is being treated with one or more other cancer treatments. Examples of such other cancer treatments include, but are not limited to, surgery, chemotherapy, checkpoint inhibitors, and radiation.

In certain embodiments, a second treatment cycle is administered to the subject if the subject has not exhibited any objective disease progression (e.g., as determined by neurologic or radiographic examination) after the treatment dose in the first treatment cycle (e.g., about 4 weeks after the treatment dose in the first treatment cycle) and has not experienced any Grade 3 or 4 adverse event (e.g., as defined by the National Cancer Institute (NCI)). A Grade 3 adverse event is generally defined as “severe and undesirable adverse event (significant symptoms requiring hospitalization or invasive intervention; transfusion; elective interventional radiological procedure; therapeutic endoscopy or operation)”. A Grade 4 adverse event is generally defined as “Life-threatening or disabling adverse event (complicated by acute, life-threatening metabolic or cardiovascular complications such as circulatory failure, hemorrhage, sepsis. Life-threatening physiologic consequences; need for intensive care or emergent invasive procedure; emergent interventional radiological procedure, therapeutic endoscopy or operation)”. Controllable fever, headache, nausea, and vomiting are not unexpected Grade 3 or 4 adverse events.

In certain embodiments, the subject receives up to about 0.5 mg, up to about 1 mg, up to about 2 mg, up to about 3 mg, up to about 4 mg, up to about 5 mg, up to about 6 mg, up to about 7 mg, up to about 8 mg, up to about 9 mg, up to about 10 mg, up to about 15 mg, or up to about 20 mg, of the anti-B7H3 antibody per treatment cycle. In certain embodiments, the subject receives at least about 0.5 mg, at least about 1 mg, at least about 2 mg, at least about 3 mg, at least about 4 mg, or at least about 5 mg, of the anti-B7H3 antibody per treatment cycle.

In certain embodiments, one treatment cycle lasts for about 1 week, about 2 weeks, about 3 weeks, about 4 weeks, about 5 weeks, about 6 weeks, about 7 weeks, about 8 weeks, about 9 weeks, or about 10 weeks. In certain embodiments, one treatment cycle lasts for about 5 weeks.

In certain embodiments, a volume of cerebrospinal fluid equal to the volume of the anti-B7H3 antibody to be injected is removed prior to administration of the anti-B7H3 antibody. In certain embodiments, the injection rate does not exceed 1 mL/min during administration of the anti-B7H3 antibody.

Dosing schedules will also vary with the disease state and status of the subject, and will typically range from a single bolus dosage or continuous infusion to multiple administrations per day, or as indicated by the treating physician and the subject's condition.

The identification of medical conditions treatable by anti-B7H3 antibodies is well within the ability and knowledge of one skilled in the art. For example, human individuals who are either suffering from primary CNS cancers or cancers metastatic to CNS are suitable for administration of an anti-B7H3 antibody. A clinician skilled in the art can readily determine, for example, by the use of clinical tests, physical examination and medical/family history, if an individual is a candidate for such treatment. In certain embodiments, the cancer is metastatic to leptomeninges. In certain embodiments, the cancer is a solid tumor.

Non-limiting examples of primary CNS cancers that can be treated with an anti-B7H3 antibody include pineoblastoma, ependymoma, medulloblastoma, chordoma, astrocytoma, glioblastoma, atypical teratoid rhabdoid tumor (ATRT), embryonal tumor with multilayered rosettes (ETMR), and choroid plexus carcinoma. In certain embodiments, the cancer is selected from the group consisting of pineoblastoma, ependymoma, medulloblastoma, chordoma, astrocytoma, and glioblastoma. In certain embodiments, the cancer is a solid tumor, for example, a pineoblastoma, ependymoma, medulloblastoma, astrocytoma, glioblastoma or chordoma.

Non-limiting examples of cancers metastatic to CNS (e.g., metastatic to leptomeninges) that can be treated with an anti-B7H3 antibody include sarcoma, retinoblastoma, melanoma, ovarian cancer, rhabdomyosarcoma, breast cancer, and lung cancer (e.g., Small cell lung cancer (SCLC) and non-small cell lung cancer (NSCLC)). In certain embodiments, the cancer is selected from the group consisting of melanoma, ovarian cancer, rhabdomyosarcoma, breast cancer, and lung cancer (e.g., Small cell lung cancer (SCLC) and non-small cell lung cancer (NSCLC)). In certain embodiments, the cancer is melanoma. In certain embodiments, the cancer is ovarian cancer.

In certain embodiments, the cancer that can be treated by the anti-B7H3 antibody is a cancer that is 8H9 reactive. 8H9 reactive cancers are disclosed in U.S. Patent Publication No. 2002/0102264, which is incorporated by reference in its entirety. 8H9 reactive cancers include, but are not limited to, cancers of varying lineage: neuroectodermal, mesenchymal and epithelial.

Any suitable method or route can be used to administer the anti-B7H3 antibody or antigen-binding fragment thereof into the CNS. In certain embodiments, the anti-B7H3 antibody or antigen-binding fragment thereof is administered intrathecally. In certain embodiments, the anti-B7H3 antibody or antigen-binding fragment thereof is administered via an intraventricular device. In certain embodiments, the intraventricular device is an intraventricular catheter. In certain embodiments, the intraventricular device is an intraventricular reservoir, for example, but not limited to, an Ommaya reservoir. In certain embodiments, the administration may be done by spinal tap or intraparenchymally.

The anti-B7H3 antibodies can be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

In certain embodiments, the subjects are administered with an additional therapeutic modality. Non-limiting examples of therapeutic modalities include chemotherapeutic agents, checkpoint inhibitor agents, and radiation therapy. In certain embodiments, the therapeutic modality is a monoclonal antibody 3F8 (MoAb 3F8), a granulocyte-macrophage-colony-stimulating factor (GM-CSF), or a combination thereof. Such one or more additional therapeutic may be administered into the CNS and/or systemically, either concurrently or sequentially with the B7H3-directed antibodies or antigen-binding fragments described herein.

### EXAMPLES

The presently disclosed subject matter will be better understood by reference to the following Example, which is provided as exemplary of the presently disclosed subject matter, and not by way of limitation.

### Example 1: Methods for Treating Neuroblastoma Metastatic to the Central Nervous System Using ¹³¹I-8H9

### Background

Neuroblastoma metastatic to the central nervous system (CNS NB) is challenging to treat and almost uniformly fatal (median survival <6 months, <10% survival at 36 months). Intraventricular compartmental radioimmunotherapy (cRIT) with the radio-iodinated monoclonal antibody ¹³¹I-8H9 offers a therapeutic strategy to eradicate NB cells in the cerebrospinal fluid.

Clinical study was conducted to demonstrate the clinical efficacy of ¹³¹I-8H9 cRIT to prolong survival of CNS NB subjects.

### Study Design and Treatment Protocol

Eligible subjects at Memorial Sloan Kettering Cancer Center (MSK) had radiographic or pathologic confirmation of CNS NB. Enrolled subjects underwent either (1) MSK temozolamide/irinotecan-based CNS salvage regimen incorporating ¹³¹I-8H9 cRIT plus systemic immunotherapy, or (2) non-regimen therapies with ¹³¹I-8H9 cRIT. Data are presented as overall survival after diagnosis of CNS metastasis.

One treatment cycle with ¹³¹I-8H9 was as follows:

Week 1: ¹³¹I-8H9 (dosimetry dose: 2 mCi imaging test dose)

Week 2: ¹³¹I-8H9 (treatment dose)

Weeks 3 and 4: observation period

Week 5: Repeated MRI of the head and spine, cerebrospinal fluid cytology

The subjects received up to two cycles of ¹³¹I-8H9 therapy. To measure the cumulative toxicity and pharmacokinetics, and to evaluate the systemic immune response to ¹³¹I-8H9 (e.g., human anti-mouse antibodies), subjects without objective disease progression (as determined by neurologic or radiographic examination) 4 weeks after the last intraventricular injection and without unexpected Grade 3 or 4 toxicity (controllable fever or headache, nausea, vomiting not included) were eligible for a second injection at the same doses administered during the first course. Subjects underwent the same treatment plan and post-treatment evaluations after the second treatment injection.

### Results

Of 105 subjects with CNS NB admitted to MSK since protocol initiation, 80 were treated with ¹³¹I-8H9 cRIT, including 57 who completed the full CNS salvage regimen. The median age of the 80 subjects that were treated with ¹³¹I-8H9 cRIT was 4.39 years.

The study's starting dose was 10 mCi ¹³¹I-8H9 per cycle for each subject and dose levels ranged to 80 mCi ¹³¹I-8H9. Eighty subjects specifically with neuroblastoma CNS/LM metastasis received doses from 10 mCi to 70 mCi. Specific activity averaged approximately 5 mCi/mg ¹³¹I-8H9 at the 10- to 50 mCi dose range, and approximately 50 mCi/mg ¹³¹I-8H9 for the 50- to 100 mCi dose range. Subjects ages 3 years or less had an adjustment to their dose.

Of 19 subjects with evaluable cancers, treatment with ¹³¹I-8H9 produced at least a partial response in 7 (36%) subjects. At analysis, 45 (56%) of the ¹³¹I-8H9-treated subjects were still alive 4.8-152 months (median 58 months) after CNS metastasis, including 36 (45%) who survived at least 36 months and 23 (29%) who survived at least 60 months. In comparison, an historic population of 19 CNS NB subjects treated at MSK before cRIT became standard care at the institution (1989-2003) survived between 2 days and 44 months (median 5.5 months) after CNS metastasis, including 2 (11%) who survived at least 36 months and none who survived beyond 44 months. Subgroup analyses of ¹³¹I-8H9-treated subjects identified age at initial NB diagnosis (<18 months) and localization of relapsed disease (isolated to CNS) as factors that positively correlated with survival; neither amplification of the MYCN oncogene, time period of enrollment in the study, nor complementary craniospinal irradiation were factors that associated with survival in these subjects.

### Conclusions

76% of subjects with CNS NB treated at MSK received ¹³¹I-8H9 cRIT, and approximately half completed an aggressive CNS salvage regimen with ¹³¹I-8H9 cRIT. Despite advanced CNS involvement, including multiple parenchymal masses with or without leptomeningeal disease in 42% of subjects, over 50% of subjects treated with ¹³¹I-8H9 cRIT are still alive and nearly 50% have survived for at least 36 months. ¹³¹I-8H9 cRIT represents a significant advancement in the treatment of CNS NB, a nearly uniformly fatal disease for which there is currently no satisfactory standard therapy.

### Example 2: Intrathecal Radioimmunotherapy Using ¹³¹I-8H9 for Central Nervous System/Leptomeningeal (CNS/LM) Neoplasms in Adult Subjects

Thirteen adult subjects older than 18 years of age and having CNS/LM neoplasms were treated with ¹³¹I-8H9 using cRIT. The average age of these thirteen adult subjects were 35.1 years, ranging from 19.4 to 53.5 years. Diagnoses of the treated thirteen adult subjects included primary CNS tumors (pineoblastoma N=1, ependymoma N=2, medulloblastoma N=3, chordoma N=1) and tumors metastatic to the CNS (melanoma N=3, rhabdomyosarcoma N=2, and ovarian cancer N=1).

Two of the 13 subjects were removed from the study after the dosimetry dose treatment, for progressive disease and compliance concerns. The rest of the 11 adult subjects were treated with ¹³¹I-8H9 cRIT according to the protocol disclosed in Example 1, with treatment doses between 10 mCi and 80 mCi. Five of the 11 subjects received a second treatment dose by undergoing a second cycle of ¹³¹I-8H9 therapy. Overall, the average dose received by the 11 subjects was about 50 mCi, and the total dose ranged from about 10 to about 160 mCi.

Reviews of MRIs after the first treatment dose showed radiographic improvement in 2 subjects (both having metastatic rhabdomyosarcoma); stable disease in three subjects; progressive disease in five subjects; and one patient had no disease at protocol entry. The average survival time following the ¹³¹I-8H9 cRIT treatment was greater than one year. Among the 11 adult subjects receiving the ¹³¹I-8H9 cRIT treatment, one subject has been alive 92 months, one alive 17 months, and two alive about 6 months, after the first injection of ¹³¹I-8H9.

Although the presently disclosed subject matter has been described in some detail by way of illustration and example for purposes of clarity of understanding, the descriptions and examples should not be construed as limiting the scope of the presently disclosed subject matter. The disclosures of all patent and scientific literature cited herein are expressly incorporated in their entireties by reference.

### Example 3: Radioimmunotherapy with Intraventricular 131I-8H9 in Patients with B7-H3 Expressing Central Nervous System Malignancies: A Phase 1/2 Study

### Introduction

Many tumors have a known predilection for the development of central nervous system (CNS) metastases. Cure remains a daunting challenge, and prognosis remains dismal despite aggressive therapy (1, 2). Brain penetration and deposition of radiolabeled tumor-specific monoclonal antibodies following convection enhanced delivery or compartmental intraventricular administration (cRIT) may overcome blood brain barrier obstacles and improve detection and treatment.(3-10) A phase 1 clinical study at Memorial Sloan Kettering Cancer Center (MSK) demonstrated the feasibility of cRIT using the anti-GD2 murine mAb 3F8 labeled with iodine-131.(11)

8H9 is a murine mAb specific for B7-H3, a surface immunomodulatory glycoprotein that is distributed on cell membranes of many pediatric and adult solid tumors.(12) When radiolabeled with iodine-124, 8H9 can be used to assess drug localization and dosimetry with positron emission tomography (PET). When radiolabeled with iodine-131, 8H9 can deliver therapeutic doses of radiation and suppresses tumors in mice.(13) The iodine-131 emits beta radiation that penetrates up to 3 mm, causing DNA damage and cell death of bound and neighboring tumor cells and tumor vasculature. cRIT

To improve the dismal prognosis of primary and metastatic tumors of the central nervous system (CNS), compartmental radioimmunotherapy (cRIT) was administered with intraventricular ¹²⁴I and ¹³¹I-labeled 8H9 targeting B7-H3 in a phase 1/2 clinical study.

To improve the dismal prognosis of primary and metastatic tumors of the central nervous system (CNS), compartmental radioimmunotherapy (cRIT) was administered with intraventricular ¹²⁴I and ¹³¹I-labeled 8H9 targeting B7-H3 in a phase 1/2 clinical study.

### Patients and Methods

Briefly, Eligibility criteria included a B7-H3 reactive CNS primary or metastatic tumor, adequate cerebrospinal fluid (CSF) flow, <grade 3 major organ toxicity, platelets >50,000/4, and ANC >1000/μL. Patients received intraventricular 2 mCi of ¹²⁴I- or ¹³¹I-8H9 for imaging and 10-to-80 mCi of ¹³¹I-8H9 for treatment. Dosimetry was based on serial CSF and blood samplings and scintigraphy over 48 hours. Follow-up magnetic resonance imaging was performed in week 5. Injections were repeated in the absence of grade 3 or 4 toxicity or progressive disease. Tumor response and overall survival (OS) were noted.

Patients with recurrent primary or metastatic CNS tumors were enrolled (2004-2017) on a MSK protocol testing ¹³¹I-8H9 cRIT (clinicalstudys.gov NCT00089245). 8H9 was purified and radiolabeled at MSK using the iodogen technique.(14) Specific activity averaged ˜5 mCi/mg ¹³¹I-8H9 at the 2-to-40 mCi dose range, and ˜50 mCi/mg ¹³¹I-8H9 for therapy doses ≥50 mCi.

Informed consent was obtained from patients or guardians. Eligibility criteria included B7-H3 reactivity of tumor by immunohistochemistry, stable neurologic status, no obstructive or symptomatic hydrocephalus, absolute neutrophil count >1000/μL, platelet count >50,000/μL, serum bilirubin <3.0 mg/dL, and serum creatinine <2 mg/dL. Prior focal or craniospinal irradiation (CSI) or chemotherapy was allowed, but not <3 weeks before enrollment. Indwelling intraventricular access devise (e.g., Ommaya catheter) position, patency and CSF flow were evaluated by pre-treatment 111-indium diethylene triamine pentaacetic acid (DTPA) studies.

### Clinical and Disease Evaluation

Pre- and post-treatment evaluation included a detailed history, physical exam, complete blood count (CBC), comprehensive profile, thyroid function tests, and CSF for total protein, glucose, cell count, cytology. All patients had baseline magnetic resonance imaging (MRI) studies of the brain and spinal cord with and without gadolinium within 3 weeks before and 1 month after cRIT. For neuroblastoma patients, staging was carried out according to the International Neuroblastoma Staging System.(15, 16) CNS neuroblastoma was defined as leptomeningeal disease or metastatic deposits in the CNS parenchyma or dura excluding skull bone-based metastases; disease evaluation for neuroblastoma patients also included ¹²³I-meta-iodobenzylguanidine (MIBG), computed tomography (CT) of the primary site, and bone marrows aspirates and biopsies. Following the completion of cRIT ¹³¹I-8H9, disease status was assessed by Mill of the brain and spine, and CSF cytology approximately every 3 months, and included CT scan of the primary site, MIBG scans, bone marrow evaluations for neuroblastoma patients.

### Dosimetry Estimates

Patients received an imaging dose of 2 mCi ¹²⁴I-8H9 or ¹³¹I-8H9, followed by CSF and blood sampling, and 3 PET or SPECT scans, respectively, over 48 hours to assess dosimetry prior to therapeutic dosing. Distribution and activity concentrations of radioactivity in the craniospinal axis, and radiation doses to plaques of disease and surrounding normal tissues were determined. ¹³¹I-8H9 radiation exposure of CSF, ventricles, spinal cord, normal brain and blood was based on the assumption of complete local absorption of the ¹³¹I beta radiation. Measured aliquots were counted to estimate the time-dependent activity concentrations. The respective time-activity data were fit to exponential functions and integrated to yield the decayed area under the curves (AUCs), representing the cumulative activity concentrations in the blood and CSF as previously described.(11)

### ¹³¹I-8H9 Therapy

To minimize thyroid uptake and to prevent a possible meningitic reaction, patients were premedicated with oral SSKI drops, Cytomel, and acetaminophen prior to injections. Dexamethasone was administered as 1 mg twice daily for 6 doses (0.5 mg for patients less than 20 kg) starting the evening before injections.

The starting dose of this phase was mCi was chosen as 10 mCi ¹³¹I-8H9 based on preclinical studies and a phase 2 study of weekly injections 10 mCi ¹³¹I-3F8 (up to 40 mCi total) can safely be administered. (24) Patients received a single dose of 10-to-80 mCi ¹³¹I-8H9, 5 mCi/mg for dose levels 1-to-4; 50 mCi/mg for dose levels 5-to-8. Dose increments were by 10 mCi every 3-6 patients. Anticipating myelosuppression in this heavily pre-treated population for doses >50 mCi, since 2009, a flat therapy dose of 50 mCi ¹³¹I-8H9 was administered on an expansion cohort. Toxicity was defined by the Common Terminology Criteria for Adverse Events (CTCAE), Version 3.0, observed in the 5 weeks after the first cycle. If toxicity grade A occurred in ≥1 of 3 patients at a given dose level, 3 more patients were accrued at that dose level. Dose limiting toxicity (DLT) was defined as toxicity grade A occurring in 2 or more of 6 patients at a level, at which maximally tolerated dose (MTD) was determined to be one dose level below the DLT. Myelosuppression due to disease or prior therapy was assessed, but not included in the assessment of MTD.

Dose adjustments based on age and corresponding CSF volume, a normal practice with intrathecally administered therapeutics, were made as follows: patients ≤12 months old received a 50% dose reduction; patients 13-to-36 months old received a 33% dose reduction; patients >36 months old received the full dose.

### Radiographical Assessment of CNS Disease

Patients were not required to have measurable disease at the time of study entry. The majority of patients lacked evaluable radiographic disease, having recently completed salvage radiation therapy, surgery, and/or chemotherapy. Radiographic images were reviewed by a board-certified MSK diagnostic neuro-radiologist. As the recent Response Assessment in Nuero-Oncology Brain Metastases (RANO-BM) initiative had not been implemented at study commencement, assessments of radiographic improvement were calculated as time to first evidence of radiographic improvement (decrease in the size of measurable parenchymal disease or improvement in enhancement for patients with leptomeningeal disease) from initial ¹³¹I-8H9 treatment²³. Swimmer plots were generated for patients with evaluable disease to display the time (in weeks) to first evidence of radiographic improvement, the length of the patient's involvement in the study or in follow-up, and survival status. Durability of response was evaluated by calculating the time to last radiographic assessment of CNS disease from initial ¹³¹I-8H9 treatment.

### Statistical Evaluations

Patients were treated on the expansion cohort based on the hypothesis and pilot data that incorporation of ¹³¹I-8H9 cRIT results in improved survival (25). Survival data were calculated from the time of diagnosis of CNS recurrence. Kaplan-Meier plots were generated to evaluate overall survival, median survival, and 95% confidence intervals (SAS, Cary, N.C.). Kaplan-Meier plots were generated on subgroups of interest in order to assess the influence of specific factors on overall survival, including histological diagnosis, age, number of prior relapses and disease status at study entry. A Mantel-Cox analysis was performed to assess the prognostic significance of: the number of injections received, total mCi ¹³¹I-8H9 delivered, and total dose of ¹³¹I-8H9 delivered to the CSF.

Historically-Treated Pediatric Patients with CNS Neuroblastoma Metastases

Nineteen patients with CNS neuroblastoma were treated at MSK before initiation of the ¹³¹I-8H9 cRIT treatment protocol. This group, and reports of survival in the literature, were used as comparator groups in the survival analysis for neuroblastoma patients treated with ¹³¹I-8H9.

### Results

Demographics and Dosing of Patients Treated with ¹³¹I-8H9 cRIT

134 patients were treated with 412 injections of cRIT ¹²⁴I-8H9 and ¹³¹I-8H9. Median age at first cRIT injection was 8.5 years (range 1.2-53.5 years). Diagnoses included metastatic CNS neuroblastoma (n=93), sarcoma (n=6), melanoma (n-4), ovarian carcinoma (n=1), and primary recurrent CNS malignancies including medulloblastoma/PNET (n=15), ependymoma (n=9), embryonal tumor with multi rosettes (n=2), rhabdoid tumor (n=1), chordoma (n=1), and choroid plexus carcinoma (n=2) (Table 1). Two patients were removed before a therapy dose was given due to progressive disease (neuroblastoma, N=1) and noncompliance (ependymoma, N=1). 37 patients were treated on the dose escalation phase 10-80 mCi ¹³¹I-8H9 (10 mCi n=7, 20 mCi n=3, 30 mCi n=6, 40 mCi n=3, 50 mCi n=3, 60 mCi n=4, 70 mCi n=6, 80 mCi n=5). The remaining 95 patients were treated at the expanded cohort flat dose level of 50 mCi ¹³¹I-8149.

### Adverse Event Profile

Patients were routinely treated awake at the bedside with direct assistance from the Pediatric, Nuclear Medicine, and Radiation Safety team in the outpatient setting. Rare grade 1 or 2 transient headache, fever, vomiting (self-limited, manageable with acetaminophen, anti-emetics) was experienced. The most common adverse event was Grade 3 or 4 myelosuppression in patients with prior craniospinal radiation, poor bone marrow reserve (<100K platelets at study entry (Table 2) and primarily observed in patients who received >50 mCi ¹³¹I-8H9. Myelosuppression was anticipated in this heavily pre-treated patient population and as such, was noted but excluded as a dose limiting toxicity. Of 132 patients, 73 (59%) received 4 injections as planned (2 dosimetry and 2 full therapy dose). Patients did not receive cycle #2 because of progressive disease (n=29), prolonged myelosuppression (n=20), acute chemical meningitis (n=3), development of subdural collections with altered CSF flow (n=2), myelodysplasia (n=2), and self-removal (n=1). Chemical meningitis was not observed in any patient on cycle #1 but was seen on retreatment after the third injection in 3 patients, manifested by acute headache, fever, vomiting and sterile CSF pleocytosis; all were self-limited events treated with supportive care and resolving over several days. No non-myelosuppressive DLT was reached (Table 2).

### Dosimetry Analysis

Eighty-nine patients had dosimetry measured by CSF sampling and ¹³¹I-8H9 SPECT; 45 patients had dosimetry measured by ¹²⁴I-8H9 PET. The change in isotope and nuclear scans was based on both isotope availability and budget constraints, with ¹²⁴I-8H9 PET being significantly costlier. A favorable therapeutic window was observed by CSF and blood samplings for both methodologies. Interpatient variability for total absorbed dose to the CSF by CSF samplings was observed; the average CSF absorbed dose for the entire cohort was 104.9 cGy/mCi compared to that in the blood 2.6 cGy/mCi. The mean total absorbed CSF dose was 3368.8 cGy (range 677-13143 cGy) by CSF sampling. The average CSF clearance was 6.7 hours. Mean total therapy dose ¹³¹I-8H9 received for the neuroblastoma patients was 67.2 mCi (19.6-104.9 mCi). 65 patients assessed for total CSF dose delivered received a total CSF dose >2100 cGy, including 24 who received only 1 therapy dose. In general, images determining region of interest were of highest quality following ¹²⁴I-8H9 PET compared to ¹³¹I-8H9 SPECT and exhibited less interpatient variability when compared to CSF sampling.

### Neuroblastoma Subgroup Analysis

93 patients with metastatic CNS neuroblastoma received 188 tracer and therapy injections, (16 dose escalation arm; 77 expanded cohort); 46 patients (50%) received a single therapy injection; 47 patients (50%) received 2 therapy injections. At the time of CNS neuroblastoma relapse, patients underwent biopsy or debulking surgery when feasible, followed by radiation therapy and chemotherapy. A Kaplan-Meier plot of overall survival for patients treated with ¹³¹I-8H9 compared with the MSK pre-cRIT CNS neuroblastoma patients is provided in FIG. 1. Patients were analyzed in 2 groups: Group 1 patients underwent full CNS and systemic directed therapy including radiation therapy, temozolamide/irinotecan, cRIT ¹³¹I-8H9, plus systemic immunotherapy using intravenous MoAb 3F8 and GMCSF as previously described (13). Group 2 patients were treated with all other therapies and cRIT ¹³¹I-8H9 (FIG. 2).

Of the 93 patients treated with ¹³¹I-8H9 cRIT, 42 (53%) remained alive from the time of CNS metastasis to the data cutoff date (range: 4.8 to 152.4 months). Of the 93 patients, 98% have survived at least 6 months, 88% have survived at least 12 months, 71% have survived at least 36 months, and 51% have survived beyond 60 months. The median survival of patients treated with ¹³¹I-8H9 cRIT was 31.8 (3.8-170.1) months (95% confidence interval [CI] lower limit: 35.2 months). In comparison, of the 19 MSK pre-cRIT patients, 6 (32%) survived at least 6 months, 4 (21%) survived at least 12 months, 2 (11%) survived beyond 36 months, and none survived to 60 months. Overall survival of the MSK control cohort ranged from 2 days to 44.1 months, with an estimated median survival of 5.5 months (95% CI: 1.1 to 8.7 months).

Eighteen of 93 patients (47%) died from causes related to a CNS recurrence of disease—either CNS recurrence alone (11 patients) or both CNS and systemic recurrence (7 patients). Sixteen patients (42%) died from recurrence of systemic disease. Four patients (11%) died from causes other than neuroblastoma. The proportion of patients whose deaths were not related to a recurrence of CNS disease (20/38, 53%) offer further evidence of the effectiveness of ¹³¹I-8H9 therapy in the treatment of CNS neuroblastoma. These patients survived up to 89.8 months after their initial ¹³¹I-8H9 treatment without recurrence of their CNS disease. Eleven survived for more than a year and four survived at least 2 years after the start of ¹³¹I-8H9 therapy.

Results for Infants with CNS Neuroblastoma

Eighteen patients with neuroblastoma initially diagnosed less than 18 months of age developed CNS metastases; 12 (66%) had MYCN amplified tumors. Patients developed CNS neuroblastoma as a site of disease recurrence (N=13) or in the setting of refractory systemic neuroblastoma (N=5). Three patients developed symptomatic neuroblastoma, progressed and died from systemic (N=2) or CNS (n=1) neuroblastoma prior to initiating cRIT131I-8H9. Of the remaining 15, 12 received the CNS salvage plan incorporating radiation therapy, chemotherapy and cRIT131I-8H9. Overall survival following cRIT 131I-8H9 was markedly prolonged for this subset of young patients compared with patients initially diagnosed at greater than 18 months of age (p=0.0096;) (FIG. 3). Twelve patients remain long-term survivors at a mean of 6.3 years (1.6-11.8 years) from the detection of CNS disease (FIG. 3), including one patient that had hundreds of neuroblastoma metastatic masses throughout the thecal space (FIG. 5).

Efficacy of ¹³¹I-8H9 cRIT Based on Radiographic Evaluation in Patients with CNS Neuroblastoma

Of the 93 patients treated with ¹³¹I-8H9 cRIT, 21 (23%) had radiographic evidence of CNS/LM disease at the time they received the initial ¹³¹I-8H9 treatment. FIGS. 4A-4B provides tabular summaries of the initial posttreatment radiographic assessment results for these patients. Of these, 9 (43%) showed radiographical improvement (decrease in size of index lesion and/or decrease in leptomeningeal enhancement) and 9 patients (43%) showed stable disease at the time of the initial radiographic assessment. Further objective evidence of the long-term clinical benefit of ¹³¹I-8H9 therapy can be provided by an analysis of the durability of radiographic stability. While the median survival for 93 patients treated with ¹³¹I-8H9 (58 months) significantly exceeds that of the historical patients (5.5 months), a significant improvement in survival may not necessarily correlate to a long-term, durable remission of disease. Treatment with ¹³¹I-8H9 induced a durable response in a significant number of patients. The median duration of radiographic response, based on available data, is 49 weeks (95% CI: 32.1-73.7 weeks) with a range of 2.6 weeks to 676 weeks (13 years). The median duration of radiographic response is likely significantly underestimated, however, as many patients have survived well beyond their last scan date. Regardless, the median duration of radiographic response to ¹³¹I-8H9 treatment (11.3 months) exceeds the historical median survival of 5.5 months (FIGS. 4A-4B).

### Subgroup Analyses and Effect on Overall Survival

There are several factors that could potentially affect survival that would not be captured in the overall survival analysis, including known demographic and genetic risk factors, disease characteristics at the time of study entry, number of relapses prior to ¹³¹I-8H9 and additional therapies received after cRIT 8H9. To determine the effect that these risk factors had on the overall survival of patients treated with ¹³¹I-8H9, subgroup analyses were performed.

For the neuroblastoma cohort, several risk factors have been consistently associated with progression of neuroblastoma and overall survival, including age at diagnosis, INSS stage, and MYCN amplification status. While age at initial diagnosis is prognostic, overall survival of patients with MYCN amplified tumors was not (p=0.2490). To determine if there was any difference in the survival of patients based on when treatment was received (i.e., the first half of the study duration [from 2004 to 2009] versus the second half [2010 to present]. There was no difference in the survival of CNS neuroblastoma patients treated during the early time period compared with the more recently treated CNS neuroblastoma patients (p=0.8851). CSI before ¹³¹I-8H9 cRIT did not have any effect on overall survival in these patients (p=0.9343). There was also no statistical difference when 21 Gy CSI vs 18 Gy CSI was administered (FIG. 6). It was also noted that no statistical difference in survival among patients receiving >50 mCi ¹³¹I-8H9 or receiving >2100 cGy to the CSF by CSF sampling. A trend towards improved survival was noted for patients receiving 2 ¹³¹I-8H9 therapy injections although not statistically significant (p=0.08).

For the other cohort of patients, prolonged survival has been observed in 6/15 patients with recurrent medulloblastoma, 3/9 patients with recurrent ependymoma, 2 patients with embryonal tumor with multilayered rosettes and 1 patient with recurrent choroid plexus carcinoma (Table 4).

Overall, 134 patients (93 neuroblastoma, 11 other non-CNS tumors, and 30 primary CNS tumors) received 412 injections. Mean absorbed dose was 104.9cGy/mCi in CSF and 2.6cGy/mCi in blood. Acute toxicities were limited. Although not a dose-limiting toxicity, grade 3 or 4 myelosuppression was noted in patients with prior craniospinal radiation therapy and at doses ≥60 mCi ¹³¹I-8H9. Improved OS was noted for the neuroblastoma cohort compared to that reported with conventional therapies.

### DISCUSSION

Targeting the B7 family of checkpoint regulators is at the forefront of cancer research, with strong evidence demonstrating a key regulatory role of B7-H3 on T-cell proliferation. B7-H3 expression is significantly associated with poor outcome in several cancers and is uniquely overexpressed in cancers compared to normal human tissues. Mature data (as long as 13 years) were presented, of a well-tolerated cRIT-based regimen incorporating compartmental radiolabeled anti-B7-H3 for incurable embryonal tumors in the pediatric population. The favorable adverse event profile, therapeutic index, and extended survival for several histologic diagnoses including recurrent CNS neuroblastoma, medulloblastoma, ependymoma, choroid plexus carcinoma and embryonal tumor with multilayered rosettes treated are tremendously encouraging. cRIT using intraventricular ¹²⁴I-8H9 and ¹³¹I-8H9 appears safe with manageable acute toxicities, even in a very young, heavily pre-treated patient population. Tumor cell cytotoxicity is attributed to the direct effect of ¹³¹I-radiation, although it is possible that a secondary mechanistic basis for successful eradication of microscopic tumor cells may in part be due to 8H9 complement activation with the CSF space. Complement components C3 and C5b-9 have been shown to be present in the CSF after intraventricular rituximab for recurrent CNS lymphoma.(17) More recent evidence suggests the diffuse tumor vasculature known overexpresses B7-H3; targeting the tumor vasculature may have additional therapeutic benefit.

The prognosis for pediatric patients diagnosed with relapsed CNS neuroblastoma, both historically and presently, given the currently available therapeutic options, remains poor. The expected survival reported across multiple sites and countries does not typically exceed 6 months and long-term survival exceeding 3 years occurs in less than 10% of patients.(18-20) The historic patient cohort from the current study was consistent with these previous studies that have reported short survival times and poor prognoses after CNS metastasis. The median overall survival of patients treated at MSK before initiation of cRIT in 2004 was 5.5 months; only 2 of the 19 patients survived at least 36 months, and none survived beyond 44 months. Regardless of therapy or the geography of the patient, these numbers have not changed significantly over the last four decades. Incorporation of cRIT ¹³¹I-8H9 represents an important advancement in the treatment of this disease, with a significant improvement in median overall survival and cure.

As the number of survivors of CNS neuroblastoma has increased over the years, it was aimed to minimize the known risks associated with craniospinal radiation in young children, most importantly neurocognitive deficits, endocrinopathies, and growth retardation. Data suggest the degree of neurocognitive impairment (i.e., mild, moderate, or severe) demonstrates dose-response patterns (11). The trend further indicates that the combination of craniospinal radiation and cRIT is able to eradicate bulky leptomeningeal deposits not amenable to surgical excision. Half of the patients in the cRIT-salvage therapy cohort were treated with 2160 cGy CSI, the standard dose administered for local control of neuroblastoma primary tumors. Since 2009, as the target cGy delivered by cRIT increased, the CSI dose was reduced to 1800 cGy, representing half of the patients in this cohort. The data demonstrated no appreciable decrease in therapeutic effect with combination external beam radiation therapy 1800 cGy and cRIT. Further, because of prior radiation therapy at initial diagnosis, extremely young age, or parental choice, 5 patients received <18 Gy CSI, yet maintained long term CNS disease control. Although only more recently pursued, proton-beam radiotherapy may be an additional way to minimize long term morbidity, delivering significantly less radiation to healthy tissue compared to conventional photon treatment. Reduced-dose craniospinal irradiation aimed at controlling bulk parenchymal and nodular leptomeningeal disease and cRIT targeting micrometastic neuroblastoma, may be sufficient to prolong survival.

An additional focus of study is determining the optimal cGy delivered to the CSF by cRIT ¹³¹I-8H9 to fully eradicate micrometastatic deposits. As patients were initially enrolled onto a phase I study with ¹³¹I-8H9, the dose of 8H9 varied. Later patients were all enrolled on an expanded phase II cohort with a uniform therapeutic dose of 50 mCi ¹³¹I-8H9. Although the average dose to bone red marrow was fairly consistent in all patients, the cGy/mCi dose delivered by cRIT to the CSF was very variable. This is likely a reflection of variable rates of CSF flow based on prior surgery, radiation, presence or absence of bulk lesions, all leading to inherent CSF flow differences prior to cRIT. As most patients were treated with cRIT as consolidation for micrometastases, the fraction of injected antibody needed to eradicate neoplastic lesions is also difficult to assess. Further, other immuno-PET studies in patients with brain metastases indicate that antibody uptake can be highly variable even in different lesions of the same patient (12). No difference was found in survival for patients receiving >50 mCi ¹³¹I-8H9, or for patients receiving >2100 cGy to the CSF by CSF sampling. This suggests that a higher dose of cRIT might not be necessary unless a lower simultaneous CSI dose is being considered.

Efforts have been made to identify which patients are at risk for the development of recurrent CNS disease. Risk factors include a diagnostic lumbar puncture at initial neuroblastoma diagnosis and MYCN amplification.(19, 20) Identifying genomic mutations driving the metastatic process has been challenging. In a SNP analysis of tumor pairs of CNS metastases and their pre-CNS primaries, only a small number of specific and recurrent differences in genomic lesions were found.(21) However, in a series of 13 CNS neuroblastoma metastases with corresponding primary tumors, the inventors previously showed miR-29a could be a biomarker for CNS metastasis; downregulation may play a pivotal role in CNS progression.(22) The known oncotargets of miR-29a included CDC6, CDK6, and DNMT3A, and B7-H3. These targets were found to have higher differential expression in brain metastases than their paired primaries.(22) Prophylactic treatment with a well-tolerated cRIT based therapy for patients at high risk for recurrence is something to be explored.

cRIT with ¹³¹I-8H9 is safe, has favorable dosimetry to CSF and blood, and shows promise for improving the prognosis of malignancies involving the CNS. Metastatic tumors to the CNS can be fully eradicated, eliminating a sanctuary site for malignancy. Intraventricular radioimmunotherapy can be successfully incorporated in curative treatment strategies for several pediatric histology including CNS neuroblastoma. These data support a role for other high-risk tumors including recurrent medulloblastoma, ependymoma, choroid plexus carcinomas, embryonal tumor with multi-layered rosettes. Overall, 60% of patients with CNS neuroblastoma metastases achieve long term remission, including 33% of patients with multiple parenchymal metastases. A survival advantage is seen for patients with B7-H3 overexpressing tumors treated with CNS directed therapy with cRIT ¹³¹I-8H9.

TABLE 1
Histologic diagnoses.

| | | No. pts on | No. pts | |
|---|---|---|---|---|
| | | phase 1 | expanded | |
| | No. | (10-80 | cohort (50 | No. |
| DIAGNOSIS | patients | mCi) | mCi) | Injections |
| Neuroblastoma | 93* | 16 | 77 | 293 |
| Medulloblastoma/ | 15 | 6 | 9 | 29 |
| PNET | | | | |
| Ependymoma | 9+ | 4 | 5 | 37 |
| ETMR | 2 | 1 | 1 | 4 |
| Sarcoma | 6 | 3 | 3 | 18 |
| Melanoma | 4 | 3 | 1 | 9 |
| Other (ATRT, | 5 | 3 | 2 | 22 |
| choroid plexus ca, | | | | |
| ovarian ca, | | | | |
| retinoblastoma) | | | | |
| TOTAL | 134** | 36 | 98 | 412 |

*One patient removed for progressive disease prior to receiving therapy injection
+One patient removed for noncompliance prior to receiving therapy injection
**132 proceeded to therapy injections

TABLE 2
Adverse event profile by histology

| | | Adverse Event | Myelosuppression |
|---|---|---|---|
| | | (CTC 3.0) | (No, %) (Gr 3 or |
| DIAGNOSIS | No. | Possibly or Probably | 4) |
| Neuroblastoma | 93 | Gr 3 or 4 | 83 (89%) |
| | | myelosuppression (ANC, | |
| | | hgb, platelets) (83) | |
| | | Gr 4 Hypersensitivity | |
| | | reaction (1) | |
| | | Gr 3 ALT/AST (5) | |
| | | Gr 3 Chemical | |
| | | Meningitis (3) | |
| | | Gr 4 MDS/AML (5) | |
| Medulloblastoma/ | 15 | Gr 3 or 4 | 6 (43%) |
| PNET | | myelosuppression (6) | |
| | | Gr 4 chemical meningitis | |
| | | (1) | |
| Ependymoma | 9 | Gr 3 or 4 | 3(33%) |
| | | myelosuppression (3) | |
| ETMR | 2 | Gr 3 or 4 | 2(100%) |
| | | myelosuppression (2) | |
| Sarcoma | 6 | Gr 3 or 4 | 3 (50%) |
| | | myelosuppression (3) | |
| | | Gr 4 AML (1) | |
| Melanoma | 4 | Gr 3 myelosuppression | 2 (50%) |
| | | (2) | |
| | | Gr 3 nausea (1) | |
| | | Gr 3 hypokalemia (1) | |
| Other (ATRT, | 5 | Gr 4 MDS/AML (1) | |
| choroid plexus | | | |
| ca, ovarian ca, | | | |
| retinoblastoma) | | | |
| TOTAL | 132 | | |

TABLE 3
Characteristics NEUROBLASTOMA Cohort.

| | | | N = 93 |
|---|---|---|---|
| Age at Initial | Median (years, range) | | 2.98 (1 day- |
| Diagnosis, years | | | 12 years) |
| Age at First cRIT | Median (years, range) | | 4.65 (16 mon- |
| Injection, years | | | 13 years) |
| Stage at Initial | 4 | | 90 |
| Diagnosis | 3, 4s | | 3 |
| MYCN- Amplified | | | 46 (49%) |
| NEUROBLASTOMA | | | |
| Craniospinal Radiation | | | |
| Treatment at CNS | | | |
| Diagnosis | | | |
| | >2160 | cGY | 3 (3%) |
| | 2160 | cGy | 30 (32%) |
| | 1800 | cGy | 44 (47%) |
| | <18 | cGy | 7 (8%) |

| | | |
|---|---|---|
| | Focal only or no CSI | 9 (10%) |
| Full CNS Radiation, | | 64 (69%) |
| Chemotherapy and | | |
| cRIT-8H9 (group 1) | | |
| cRIT-8H9 and all other | | 29 (31%) |
| therapies (group 2) | | |
| # Relapses prior to | 0 | 55 (59%) |
| cRIT 8H9 | 1 | 18 (19%) |
| | 2 | 1 (1%) |
| | 3 | 0 |
| | 4 | 1 (1%) |
| | Refractory systemic | 17 (18%) |
| Type of CNS disease | Unifocal Parenchymal | 54 (58%) |
| | Mass | |
| | Multifocal Parenchymal | 21 (23%) |
| | Masses | |
| | Leptomeningeal | 9 (10%) |
| | Parenchymal + | 9 (10%) |
| | Leptomeningeal | |
| Symptomatic CNS | | 59 (63%) |
| NEUROBLASTOMA | | |
| Status when treated | Stable Evaluable Disease | 21 (23%) |
| with cRIT 8H9: | Radiographic/Cytologic | 72 (77%) |
| | Remission | |

Numbers represent frequencies with percents in parentheses unless otherwise specified

TABLE 4
Survival for Other Embryonal and Other CNS Malignancies

| | | Overall Survival |
|---|---|---|
| Diagnosis | No. Patients | (mon) |
| Medulloblastoma | 15 | 8.2 (1-100) |
| Ependymoma | 9 | 13.3 (2.8-117) |
| ETMR | 2 | 34 (2053) |
| Sarcoma | 6 | 10 (0.7-46) |
| Melanoma | 4 | 5 (0.6-7.3) |
| Other (ATRT, CPP, | 5 | 9.3-98 |
| Ovarian Ca, RB, | | |
| chordoma) | | |

### REFERENCES

1. Wiens A L, Hattab E M. The pathologic spectrum of solid CNS metastases in the pediatric population. J Neurosurg Pediatr 2014, 14 (2): 129-135.
2. De B, Kinnaman M D, Wexler L H, Kramer K, Wolden S L. Central nervous system relapse of rhabdomyosarcoma. Pediatr Blood Cancer. 2018 January; 65(1). doi: 10.1002/pbc.26710. Epub 2017 Jul. 11.
3. Pizzo M E, Wolak D J, Kumar N N, Brunette E, Brunnquell C L, Hannocks M J, Abbott N J, Meyerand M E, Sorokin L, Stanimirovic D B, Thorne R G. Intrathecal antibody distribution in the rat brain: surface diffusion, perivascular transport and osmotic enhancement of delivery. J Physiol. 2018 Feb. 1; 596(3):445-475. doi: 10.1113/JP275105. Epub 2017 Dec. 18
4. Convection-Enhanced Delivery for Diffuse Intrinsic Pontine Glioma Treatment. Zhou Z, Singh R, Souweidane M M. Curr Neuropharmacol. 2017; 15(1):116-128.
5. Kramer K, Smith-Jones P M, Humm J L, Zanzonico P, Pandit-Taskar N, Carrasquillo J, Modak S, Tickoo S, Gerald W L, Dunkel I J, Khakoo Y, Gilheeney S W, Souweidane M M, Larson S M, and Cheung N K V. Radioimmunotherapy for High-Risk and Recurrent Central Nervous System (CNS) Cancers: Results of a Phase II Study with intra-Ommaya ¹³¹I-3F8. NeuroOncology 2010; 12(6): ii48
6. Kramer K, Kushner B H, Modak S, Pandit-Taskar N, Smith-Jones P, Zanzonico P, Humm J L, Xu H, Wolden S L, Souweidane M M, Larson S M, Cheung N K. Compartmental intrathecal radioimmunotherapy: results for treatment for metastatic CNS neuroblastoma. J Neurooncol 2010; 97(3):409-187.
7. Mehta A I, Choi B D, Ajay D, Raghavan R, Brady M, Friedman A H, Pastan I, Bigner D D, Sampson J H. Convection enhanced delivery of macromolecules for brain tumors. Curr Drug Discov Technol. 2012 December; 9(4):305-10.
8. Pizer B L, Papanastassiou V, Moseley R, Tzanis S, Hancock J P, Kemshead J T, et al. Meningeal Leukemia and Medulloblastoma—Preliminary Experience with Intrathecal Radioimmunotherapy. Antibody Immunoconj. 1991; 4(4):753-61.
9. Bigner D D, Brown M T, Friedman A H, Coleman R E, Akabani G, Friedman H S, et al. Iodine-131-labeled antitenascin monoclonal antibody 8106 treatment of patients with recurrent malignant gliomas: phase I study results. Journal of clinical oncology: official journal of the American Society of Clinical Oncology. 1998; 16(6):2202-12.
10. Cokgor I, Akabani G, Friedman H S, Friedman A H, Zalutsky M R, Zehngebot L M, et al. Long term response in a patient with neoplastic meningitis secondary to melanoma treated with (131)I-radiolabeled antichondroitin proteoglycan sulfate Mel-14 F(ab′)(2): a case study. Cancer. 2001; 91(9):1809-13.
11. Kramer K, Humm J L, Souweidane M M, Zanzonico P B, Dunkel U, Gerald W L, et al. Phase I study of targeted radioimmunotherapy for leptomeningeal cancers using intra-Ommaya 131-I-3F8. Journal of clinical oncology: official journal of the American Society of Clinical Oncology. 2007; 25(34):5465-70.
12. Modak S, Kramer K, Gultekin S H, Guo H F, Cheung N K. Monoclonal antibody 8H9 targets a novel cell surface antigen expressed by a wide spectrum of human solid tumors. Cancer Res. 2001; 61(10):4048-54.
13. Modak S, Guo H F, Humm J L, Smith-Jones P M, Larson S M, Cheung N K. Radioimmunotargeting of human rhabdomyosarcoma using monoclonal antibody 8H9. Cancer Biother Radiopharm. 2005; 20(5):534-46.
14. Miraldi F D, Nelson A D, Kraly C, Ellery S, Landmeier B, Coccia P F, et al. Diagnostic imaging of human neuroblastoma with radiolabeled antibody. Radiology. 1986; 161(2):413-8.
15. Brodeur G M, Seeger R C, Barrett A, Berthold F, Castleberry R P, D'Angio G, et al. International criteria for diagnosis, staging, and response to treatment in patients with neuroblastoma. J Clin Oncol. 1988; 6(12):1874-81.
16. Monclair T, Brodeur G M, Ambros P F, Brisse H J, Cecchetto G, Holmes K, et al. The International Neuroblastoma Risk Group (INRG) Staging System: An INRG Task Force Report. Journal of Clinical Oncology. 2009; 27(2):298-303.
17. Kadoch C, Li J, Wong V S, Chen L, Cha S, Munster P, et al. Complement activation and intraventricular rituximab distribution in recurrent central nervous system lymphoma. Clinical cancer research: an official journal of the American Association for Cancer Research. 2014; 20(4):1029-41.
18. Kellie S J, Hayes F A, Bowman L, Kovnar E H, Langston J, Jenkins J J, 3rd, et al.

Primary extracranial neuroblastoma with central nervous system metastases characterization by clinicopathologic findings and neuroimaging. Cancer. 1991; 68(9):1999-2006.
19. Kramer K, Kushner B, Heller G, Cheung N K. Neuroblastoma metastatic to the central nervous system. The Memorial Sloan-kettering Cancer Center Experience and A Literature Review. Cancer. 2001; 91(8):1510-9.
20. Matthay K K, Brisse H, Couanet D, Couturier J, Benard J, Mosseri V, et al. Central nervous system metastases in neuroblastoma: radiographic, clinical, and biologic features in 23 patients. Cancer. 2003; 98(1):155-65.
21. Cobrinik D, Ostrovnaya I, Hassimi M, Tickoo S K, Cheung I Y, Cheung N K. Recurrent pre-existing and acquired DNA copy number alterations, including focal TERT gains, in neuroblastoma central nervous system metastases. Genes, chromosomes & cancer. 2013; 52(12):1150-66.
22. Cheung I Y, Farazi T A, Ostrovnaya I, Xu H, Tran H, Mihailovic A, et al. Deep MicroRNA sequencing reveals downregulation of miR-29a in neuroblastoma central nervous system metastasis. Genes, chromosomes & cancer. 2014; 53(10):803-14.
23. Alexander B M, Brown P D, Ahluwalia M S, Aoyama H, Baumert B G, Chang S M, Gaspar L E, Kalkanis S N, Macdonald D R, Mehta M P, Soffietti R, Suh J H, van den Bent M J, Vogelbaum M A, Wefel J S, Lee E Q, Wen P Y; Response Assessment in Neuro-Oncology (RANO) group. Clinical trial design for local therapies for brain metastases: a guideline by the Response Assessment in Neuro-Oncology Brain Metastases working group. Lancet Oncol. 2018 January; 19(1):e33-e42. doi: 10.1016/S1470-2045(17)30692-7.
24. Kramer K, Pandit-Taskar N, Humm J L, Zanzonico P B, Haque S, Dunkel U, Wolden S L, Donzelli M, Goldman D A, Lewis J S, Lyashchenko S K, Khakoo Y, Carrasquillo J A, Souweidane M M, Greenfield J P, Lyden D, De Braganca K D, Gilheeney S W, Larson S M, Cheung N K V. A phase II study of radioimmunotherapy with intraventricular 131I-3F8 for medulloblastoma. Pediatr Blood Cancer. 2017 Sep. 22. doi: 10.1002/pbc.26754. (PMID: 28940863)
25. Kramer K, Kushner B H, Modak S, Pandit-Taskar N, Smith-Jones P, Zanzonico P, Humm J L, Xu H, Wolden S L, Souweidane M M, Larson S M, Cheung N K. Compartmental intrathecal radioimmunotherapy: results for treatment for metastatic CNS neuroblastoma. Journal of Neurooncology, 97:409-418, 2010. (PMID: 19890606)

What is claimed is:

1. A method for treating a central nerve system (CNS) cancer in a human subject, comprising administering into the CNS of the subject a therapeutically effective amount of an antibody or an antigen-binding fragment thereof that specifically binds to human B7H3, wherein the cancer is a primary central nerve system (CNS) cancer or a cancer metastatic to CNS, and the antibody or antigen-binding fragment thereof is conjugated to a radioactive isotope and/or a therapeutic modality.

2. The method of claim 1, wherein the human subject is an adult.

3. The method of claim 1 or 2, wherein the cancer is metastatic to leptomeninges.

4. The method of any one of claim 1-3, wherein the cancer metastatic to CNS is a non-CNS solid tumor.

5. The method of claim 4, wherein the solid tumor is selected from the group consisting of sarcoma, melanoma, ovarian cancer, and rhabdomyosarcoma.

6. The method of claim 5, wherein the solid tumor is selected from the group consisting of melanoma, ovarian cancer, and rhabdomyosarcoma.

7. The method of any one of the claims 1-6, wherein the central nerve system (CNS) cancer is selected from the group consisting of neuroblastoma and primary recurrent CNS malignancies.

8. The method of any one of claims 1-7, wherein the antibody or antigen-binding fragment thereof is selected from the group consisting of murine antibodies or antigen-binding fragments thereof, humanized antibodies and antigen-binding fragments thereof, chimeric antibodies and antigen-binding fragments thereof, and human antibodies and antigen-binding fragments thereof.

9. The method of claim 8, wherein the antibody or antigen-binding fragment thereof is a murine antibody or an antigen-binding fragment thereof.

10. The method of any one of claims 1-9, wherein the antibody or antigen-binding fragment thereof binds to FG-loop of B7H3.

11. The method of any one of claims 1-10, wherein the antibody or antigen-binding fragment thereof comprises:
(a) a heavy chain variable region CDR1 comprising the amino acid sequence set forth in SEQ ID NO: 3,
(b) a heavy chain variable region CDR2 comprising the amino acid sequence set forth in SEQ ID NO: 4,
(c) a heavy chain variable region CDR3 comprising the amino acid sequence set forth in SEQ ID NO: 5,
(d) a light chain variable region CDR1 comprising the amino acid sequence set forth in SEQ ID NO: 6,
(e) a light chain variable region CDR2 comprising the amino acid sequence set forth in SEQ ID NO: 7, and
(f) a light chain variable region CDR3 comprising the amino acid sequence set forth in SEQ ID NO: 8.

12. The method of any one of claims 1-11, wherein the antibody or antigen-binding fragment thereof comprises:
(a) a heavy chain variable region comprising the amino acid sequence set forth in SEQ ID NO: 1, and
(b) a light chain variable region comprising the amino acid sequence set forth in SEQ ID NO: 2.

13. The method of any one of claims 1-12, wherein the antibody or antigen-binding fragment thereof is administered intrathecally to the subject.

14. The method of any one of claims 1-13, wherein the antibody or antigen-binding fragment thereof is administered to the subject via an intraventricular device.

15. The method of claim 14, wherein the intraventricular device is an intraventricular catheter.

16. The method of claim 14, wherein the intraventricular device is an intraventricular reservoir.

17. The method of any one of claims 1-16, wherein the radioactive isotope is ¹²⁴I, ¹³¹I, ¹⁷⁷Lu, or ⁹⁹mTc.

18. The method of any one of claims 1-17, comprising administering to the subject one treatment cycle of the antibody or antigen-binding fragment thereof.

19. The method of any one of claims 1-18, comprising administering to the subject two treatment cycles of the antibody or antigen-binding fragment thereof.

20. The method of claim 18 or 19, wherein one treatment cycle comprises a dosimetry dose and a treatment dose.

21. The method of any one of claims 1-20, wherein the therapeutically effective amount is from about 10 mCi to about 200 mCi or from about 10 mCI to about 100 mCi.

22. The method of any one of claims 1-21, wherein the therapeutically effective amount is about 50 mCi.

23. The method of any one of claims 1-22, wherein the method prolongs survival of the subject.

24. The method of any one of claims 1-23, wherein the method prolongs remission of the cancer in the subject.

25. The method of any one of claims 1-24, wherein the antibody or antigen-binding fragment thereof comprises an amino acid sequence having at least about 80%, about 90%, about 95%, about 99% or about 100% homologous to the amino acid sequence set forth in SEQ ID NO: 17.

26. The method of any one of claims 1-25, wherein the antibody or antigen-binding fragment thereof comprises the amino acid sequence set forth in SEQ ID NO: 17.

27. The method of any one of claims 1-26, wherein the antibody or antigen-binding fragment thereof has amino acids 224-241 of SEQ ID NO: 17.

28. The method of any one of claims 1-26, wherein the antibody or antigen-binding fragment thereof has amino acids 242-267 of SEQ ID NO: 17.

29. The method of any one of claims 1-28, wherein the therapeutic modality is selected from the group consisting of one or more chelator compound, one or more chemotherapeutic agent, one or more checkpoint inhibitor agent, and radiation therapy.

30. The method of claim 29, wherein the therapeutic modality is a chelator compound.

31. The method of claim 29 or 30, wherein the antibody or antigen-binding fragment thereof is conjugated to a chelator compound, wherein the chelator compound is bound to a radioactive isotope.

32. The method of any one of claims 29-31, wherein the chelator compound is DOTA or DTPA.

33. The method of claim 29, wherein the therapeutic modality is a monoclonal antibody 3F8 (MoAb 3F8), a granulocyte-macrophage-colony-stimulating factor (GM-CSF), or a combination thereof.

34. The method of any one of claims 1-33, wherein the therapeutic modality is administered into the CNS of the subject and/or systemically to the subject.

35. The method of any one of claims 1-34, wherein the therapeutic modality is administered to the subject concurrently or sequentially with the antibody or antigen-binding fragment thereof.

36. An antibody or an antigen-binding fragment thereof binding specifically to human B7H3, wherein the antibody or antigen-binding fragment thereof is conjugated to a chelator compound, wherein the chelator compound is bound to a radioactive isotope.

37. The antibody or antigen-binding fragment thereof of claim 36, wherein the antibody or antigen-binding fragment thereof is selected from the group consisting of murine antibodies and antigen-binding fragments thereof, humanized antibodies and antigen-binding fragments thereof, chimeric antibodies and antigen-binding fragments thereof, and human antibodies and antigen-binding fragments thereof.

38. The antibody or antigen-binding fragment thereof of claim 36 or 37, wherein the antibody or antigen-binding fragment thereof is a murine antibody or an antigen-binding fragment thereof.

39. The antibody or antigen-binding fragment thereof of any one of claims 36-38, wherein the antibody or antigen-binding fragment thereof binds to FG-loop of B7H3.

40. The antibody or antigen-binding fragment thereof of any one of claims 36-39, wherein the antibody or antigen-binding fragment thereof comprises:
a. a heavy chain variable region CDR1 comprising the amino acid sequence set forth in SEQ ID NO: 3,
b. a heavy chain variable region CDR2 comprising the amino acid sequence set forth in SEQ ID NO: 4,
c. a heavy chain variable region CDR3 comprising the amino acid sequence set forth in SEQ ID NO: 5,
d. a light chain variable region CDR1 comprising the amino acid sequence set forth in SEQ ID NO: 6,
e. a light chain variable region CDR2 comprising the amino acid sequence set forth in SEQ ID NO: 7, and
f. a light chain variable region CDR3 comprising the amino acid sequence set forth in SEQ ID NO: 8.

41. The antibody or antigen-binding fragment thereof of any one of claims 36-40, wherein the antibody or antigen-binding fragment thereof comprises:
(a) a heavy chain variable region comprising the amino acid sequence set forth in SEQ ID NO: 1, and
(b) a light chain variable region comprising the amino acid sequence set forth in SEQ ID NO: 2.

42. The antibody or antigen-binding fragment thereof of any one of claims 36-41, wherein the radioactive isotope is ¹²⁴I, ¹³¹I, ¹⁷⁷Lu, or ⁹⁹mTc.

43. The antibody or antigen-binding fragment thereof of any one of claims 36-42, wherein the chelator compound is DOTA or DTPA

44. The antibody or antigen-binding fragment thereof of any one of claims 36-43, wherein the antibody or antigen-binding fragment thereof comprises an amino acid sequence having at least about 80%, about 90%, about 95%, about 99% or about 100% homologous to the amino acid sequence set forth in SEQ ID NO: 17.

45. The antibody or antigen-binding fragment thereof of any one of claims 36-44, wherein the antibody or antigen-binding fragment thereof comprises the amino acid sequence set forth in SEQ ID NO: 17.

46. The antibody or antigen-binding fragment thereof of any one of claims 36-45, wherein the antibody or antigen-binding fragment thereof has amino acids 224-241 of SEQ ID NO: 17.

47. The antibody or antigen-binding fragment thereof of any one of claims 36-46, wherein the antibody or antigen-binding fragment thereof has amino acids 242-267 of SEQ ID NO: 17.

48. The antibody or antigen-binding fragment thereof of any one of claims 36-47, wherein the antibody or antigen-binding fragment thereof is a DOTA-8H9 conjugate or a DTPA-8H9 conjugate.

49. The antibody or antigen-binding fragment thereof of any one of the claims 36-48, wherein the antibody or antigen-binding fragment thereof is a ¹⁷⁷Lu-DOTA-8H9 conjugate or a ¹⁷⁷Lu-DTPA-8H9 conjugate or (177)LU-CHX-A″-DTPA-8H9.

50. The antibody or antigen-binding fragment thereof of any one of claims 36-49, wherein the antigen-binding fragment thereof is a single chain variable fragment (scFv).

51. The antibody or antigen-binding fragment thereof of claim 50, wherein the scFv comprises a portion of the amino acid sequence set forth in SEQ ID NO: 9, SEQ ID NO: 13, and SEQ ID NO: 14.

52. A composition comprising the antibody or antigen-binding fragment thereof of any one of claims 36-51.

53. A pharmaceutical composition comprising the antibody or antigen-binding fragment thereof of any one of claims 36-51, and a pharmaceutically acceptable carrier.

54. A method for imaging a tumor in a subject comprising administering to the subject an antibody or antigen-binding fragment thereof of any one of claims 36-51.

55. An antibody or antigen-binding fragment thereof of any one of claims 36-51 for use as a medicament.

56. An antibody or antigen-binding fragment thereof of any one of claims 36-51 for use in the treatment of cancer.

57. An antibody or antigen-binding fragment thereof of any one of claims 36-51 for use in the treatment of a central nerve system (CNS) cancer.

58. An antibody or antigen-binding fragment thereof of any one of claims 36-51 for use in the treatment of metastatic CNS neuroblastoma, sarcoma, melanoma, ovarian carcinoma, and primary recurrent CNS malignancies.

59. An antibody or antigen-binding fragment thereof of any one of claims 36-51 for use in a method for imaging a tumor in a subject.

60. An antibody or antigen-binding fragment thereof of any one of claims 36-51 for use in a method according to any one of claims 1-35.

61. Use of an antibody or antigen-binding fragment thereof of any one of claims 36-51 for the preparation of a medicament for killing and/or reducing tumor cells and/or inhibiting growth of the tumor.

62. Use of an antibody or antigen-binding fragment thereof of any one of claims 36-51 for the preparation of a medicament for imaging tumor cells bearing the antigen recognized by the antibody or antigen-binding fragment thereof.

63. Use of an antibody or antigen-binding fragment thereof of any one of claims 36-51 for the preparation of a medicament for a method according to any of claims 1-35.


SEQUENCE LISTING (17 sequences)

SEQ ID NO: 1 - protein, 442 aa, Mus musculus
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 214 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 5 aa, Mus musculus
NYDIN

SEQ ID NO: 4 - protein, 11 aa, Mus musculus
WIFPGDGSTQY

SEQ ID NO: 5 - protein, 9 aa, Mus musculus
QTTATWFAY

SEQ ID NO: 6 - protein, 11 aa, Mus musculus
RASQSISDYLH

SEQ ID NO: 7 - protein, 7 aa, Mus musculus
YASQSIS

SEQ ID NO: 8 - protein, 9 aa, Mus musculus
QNGHSFPLT

SEQ ID NO: 9 - protein, 243 aa, Mus musculus
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSGGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 10 - DNA, 731 bp, Mus musculus
caggtcaaactgcagcagtctggggctgaactggtaaagcctggggcttcagtgaaattgtcctgcaaggcttctggctacaccttcacaaactatgatataaactgggtgaggcagaggcctgaacagggacttgagtggattggatggatttttcctggagatggtagtactcaatacaatgagaagttcaagggcaaggccacactgactacagacacatcctccagcacagcctacatgcagctcagcaggctgacatctgaggactctgctgtctatttctgtgcaagacagactacggctacctggtttgcttactggggccaagggaccacggtcaccgtctcctcaggtggaggcggttcaggcggaggtggctctggcggtggcggatcggacatcgagctcactcagtctccaaccaccctgtctgtgactccaggagatagagtctctctttcctgcagggccagccagagtattagcgactacttacactggtaccaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaacaggcggccgc

SEQ ID NO: 11 - DNA, 731 bp, Mus musculus
gtccagtttgacgtcgtcagaccccgacttgaccatttcggaccccgaagtcactttaacaggacgttccgaagaccgatgtggaagtgtttgatactatatttgacccactccgtctccggacttgtccctgaactcacctaacctacctaaaaaggacctctaccatcatgagttatgttactcttcaagttcccgttccggtgtgactgatgtctgtgtaggaggtcgtgtcggatgtacgtcgagtcgtccgactgtagactcctgagacgacagataaagacacgttctgtctgatgccgatggaccaaacgaatgaccccggttccctggtgccagtggcagaggagtccacctccgccaagtccgcctccaccgagaccgccaccgcctagcctgtagctcgagtgagtcagaggttggtgggacagacactgaggtcctctatctcagagagaaaggacgtcccggtcggtctcataatcgctgatgaatgtgaccatggttgtttttagtgtactcagaggttccgaagagtagtttatacgaagggttaggtagagaccctaggggaggtccaagtcaccgtcacctagtcccagtctaaagtgagagtcatagttgtcacaccttggacttctacaacctcacataatgacagttttaccagtgtcgaaaggcgagtgcaagccacgaccctggttcgacctcgactttgtccgccggcg

SEQ ID NO: 12 - DNA, 731 bp, Mus musculus
caggtcaaactgcagcagtctggggctgaactggtaaagcctggggcttcagtgaaattgtcctgcaaggcttctggctacaccttcacaaactatgatataaactgggtgaggcagaggcctgaacagggacttgagtggattggatggatttttcctggagatggtagtactcaatacaatgagaagttcaagggcaaggccacactgactacagacacatcctccagcacagcctacatgcagctcagcaggctgacatctgaggactctgctgtctatttctgtgcaagacagactacggctacctggtttgcttactggggccaagggaccacggtcaccgtctcctcagatggaggcggttcaggcggaggtggctctggcggtggcggatcggacatcgagctcactcagtctccaaccaccctgtctgtgactccaggagatagagtctctctttcctgcagggccagccagagtattagcgactacttacactggtaccaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaacaggcggccgc

SEQ ID NO: 13 - protein, 243 aa, Mus musculus
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 14 - protein, 243 aa, Mus musculus
QVKLQQSGAELVEPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDQVSLSCRASQSISDYLHWYQQKSHESPQLLIKYASQSISGIPSRFSGSGSGSDFTLSINSVEPEDVGVYYCQNGHSFPLTFGAGTELELEQAA

SEQ ID NO: 15 - protein, 18 aa, Mus musculus
NPVLQQDAHSSVTITPQR

SEQ ID NO: 16 - protein, 26 aa, Mus musculus
SPTGAVEVQVPEDPVVALVGTDATLR

SEQ ID NO: 17 - protein, 534 aa, Mus musculus
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA